(19) United States
(12) Patent Application Publication
(10) Pub. No.: US 2020/0202977 A1
(43) Pub. Date: Jun. 25, 2020

(54) SEQUENCING SYSTEM WITH MULTIPLEXED BIOLOGICAL SAMPLE AGGREGATION

(71) Applicant: Illumina, Inc., San Diego, CA (US)

(72) Inventors: Eric Smith, San Diego, CA (US); James Bierle, San Diego, CA (US); Sean Kim, San Diego, CA (US); Tyler Aragon, San Diego, CA (US); Pedro Cruz, San Diego, CA (US); Rodger Constandse, San Diego, CA (US)

(73) Assignee: Illumina, Inc., San Diego, CA (US)

(21) Appl. No.: 16/614,339

(22) PCT Filed: Jul. 25, 2018
(86) PCT No.: PCT/US2018/043744

§ 371 Date: Nov. 15, 2019

Related U.S. Application Data
(60) Provisional application No. 62/539,402, filed on Jul. 31, 2017.

(51) Int. Cl. G16B 30/00; C12Q 1/6869

(52) U.S. Cl. CPC G16B 30/00; C12Q 1/6869

(57) ABSTRACT

A wide variety of scenarios are supported for per-biosample aggregation of sequencing yield. A sequencing system can sequence a plurality of biosamples in parallel. As sequencing yield results are acquired, they can be matched to biosamples and sequencing progress for the biosamples can be monitored. A target amount of yield can be specified so that a sequencing yield analysis application is automatically launched when aggregated yield reaches the target. Other features related to quality control and yield-in-progress can result in more efficient sequencing activities and reduction in waste.

### CROSS REFERENCE TO RELATED APPLICATION

This application claims the benefit of U.S. Provisional Application No. 62/539,402, filed on Jul. 31, 2017, which is hereby incorporated herein by reference.

### BACKGROUND

Sequencing technology continues to advance at an incredible rate. What once took months or years to accomplish can now be accomplished in a few days. However, while the ability to complete sequencing tasks has advanced, the logistics of coordinating such tasks have now progressed beyond the ability of the tools that are available to the lab or scientist. For example, in a high-throughput laboratory environment, dozens of sequencing tasks can be run in parallel. Due to the availability of multiplexed sequencing runs, it is possible to run a large number of sequencing tasks in parallel on a single sequencing machine. On top of such complexities, it is common practice to run a number of sequencing machines at the same time in a single lab.

Therefore, as multiplexing and other technologies have provided a more efficient and faster sequencing environment, the ability to generate sequencing data has outpaced the ability to synthesize and analyze the resulting sequencing data.

There is therefore room for improvement.

### SUMMARY

The Summary is provided to introduce a selection of concepts in a simplified form that are further described below in the Detailed Description. The Summary is not intended to identify key features or essential features of the claimed subject matter, nor is it intended to be used to limit the scope of the claimed subject matter.

In one embodiment, a sequencing device system comprises a plurality of sequencing devices that output multiplexed raw biosample sequencing data for a plurality of input biosamples comprising a particular biosample, wherein a target number of base pairs of sequence yield is specified as sufficient for launching an application for further analysis of the particular biosample; one or more processors; and memory coupled to the one or more processors, wherein the memory comprises computer-executable instructions causing the one or more processors to perform a process comprising: receiving, from the plurality of sequencing devices, the multiplexed raw biosample sequencing data for the plurality of input biosamples; demultiplexing and converting the multiplexed raw biosample sequencing data into a plurality of candidate biosample sequencing yield data sets; identifying which of the candidate biosample sequencing yield data sets originates from the particular biosample; aggregating the candidate biosample sequencing yield data sets originating from the particular biosample into aggregated sequencing data yield for the particular biosample; determining whether the aggregated sequencing data yield for the particular biosample is sufficient, wherein determining whether the aggregating sequencing data yield is sufficient comprises comparing a number of base pairs in the aggregated sequencing data yield for the particular biosample to the target number of base pairs; and responsive to determining that the aggregated sequencing data yield for the particular biosample is sufficient, launching an application performing the further analysis of the particular biosample with the aggregated sequencing data yield for the particular biosample.

In another embodiment, a sequencing device system comprises a plurality of sequencing devices that output multiplexed raw biosample sequencing data for a plurality of input biosamples comprising a particular biosample; in one or more computer-readable media, internal representations of sequencing runs, lanes, libraries, and biosamples stored as run identifiers, lane identifiers, library identifiers, and biosample identifiers; and a yield aggregator configured to receive a demultiplexed candidate biosample sequencing yield data set originating from the multiplexed raw biosample sequencing data, determine, from the internal representations, that the data set originates from the particular biosample, aggregate the data set with other data sets originating from a same particular biosample, and provide an indication of total amount of yield acquired for the particular biosample.

As described herein, a variety of other features and advantages can be incorporated into the technologies as desired.

### BRIEF DESCRIPTION OF THE DRAWINGS

FIG. 1 is a block diagram of an example system implementing multiplexed biological sample aggregation.

FIG. 2 is a flowchart of an example method implementing multiplexed biological sample aggregation.

FIG. 3 is a block diagram of an example system performing a single sequencing run for use in multiplexed biological sample aggregation.

FIG. 4 is a flowchart of an example method of performing a single sequencing run for use in multiplexed biological sample aggregation.

FIG. 5 is a block diagram of example relationships for sequencing entities in multiplexed biological sample aggregation scenarios.

FIG. 6 is a flowchart of an example method of processing sequencing entities in multiplexed biological sample aggregation scenarios.

FIG. 7 is a block diagram of an example system aggregating yield from multiplexed biological samples.

FIG. 8 is a flowchart of an example method of aggregating yield from multiplexed biological samples.

FIG. 9 is a block diagram of an example system selectively aggregating yield from multiplexed biological samples based on quality control.

FIG. 10 is a flowchart of an example method of implementing quality-control-based selective aggregation.

FIG. 11 is a block diagram of an example aggregation system showing details of how data relating to a particular biosample is identified as originating from a particular biosample.

FIG. 12 is a flowchart of an example aggregation method of showing details of how data relating to a particular biosample is identified as originating from a particular biosample.

FIG. 13 is a block diagram of an example system tracking yield progress via a quality-control-based selective yield aggregator.

FIG. 14 is a flowchart of an example method of tracking yield progress in a quality-control-based selective yield aggregation scenario.

FIG. 15 is a flowchart of an example method of determining whether there is sufficient sequencing yield for a biosample, accounting for yield-in-progress.

FIGS. 16A-D are bar graphs showing yield progress in an example quality-control-based selective yield aggregation scenario involving quality control failure.

FIG. 17 shows an internal representation of yield progress in an example quality-control-based selective yield aggregation scenario involving quality control failure.

FIGS. 18A-E and 19A-D are bar graphs showing yield progress in an example expired yield scenario.

FIG. 20 is a block diagram of an example system matching expected yield from sequencing runs to lab requests for tracking yield progress.

FIG. 21 is a flowchart of an example method of matching expected yield from sequencing runs to lab requests for tracking yield progress.

FIG. 22 is a block diagram of an example internal representation of relationships between sequencing entities for use during yield matching.

FIG. 23 is a flowchart of a method of an example implementation of the technologies into a comprehensive sequencing solution.

FIG. 24 is a flowchart of an example method of implementing work orders for the technologies.

FIG. 25 is a flowchart of an example method of implementing quality control in a sequencing data aggregation scenario by sequencing lane.

FIG. 26 is a flowchart of an example method of implementing quality-control-based selective yield aggregation across sequencing entities.

FIG. 27 is a diagram of an example computing system in which described embodiments can be implemented.

### DETAILED DESCRIPTION

A variety of scenarios involving aggregation of sequencing data by biosample are described herein. Sequencing yield originating from a variety of sequencing entities can be aggregated together under a variety of circumstances, providing more efficient sequencing data processing and faster results. Other features can be incorporated to enhance the technologies as described herein.

Quality control can be automated to implement selective aggregation so that aggregation results provide meaningful, usable information that can be used to decide when further analysis can continue.

Automatic launching of an application to perform further analysis on aggregated yield can be triggered when aggregation results indicate that sufficient yield has been aggregated.

As described herein, yield-in-progress features can help avoid false positives in a missing yield determination. As a result, wasted sequencing runs and excessive over sequencing can be avoided.

The technologies can account for failed yield, such as that related to failed quality control metrics. A requeue alert can be provided so that sufficient yield can be acquired in a timely manner. The technologies can account for such requeues in missing yield determinations. Timeouts can be used to implement an expired yield scenario.

Scientists can benefit from the technologies because accurate aggregated yield can better indicate missing yield, failed yield, and the like. Automatic launching of further analysis applications can lead to significantly higher throughput due to the amount of time needed to complete such further analysis.

Therefore, overall performance of sequencing and related analysis can be enhanced as described herein.

### Example 1—Example Advantages

As described herein, a number of advantages can result from the technologies. In some cases, the bottleneck for completing analysis of a biosample can be determining that there is enough yield. Due to the multiplexed nature of sequencing, it is not immediately apparent that a completed sequencing run indicates that there is now sufficient yield, and that further analysis can be initiated. Because such further analysis can take significant time to complete, the technologies can greatly improve overall throughput by automatically launching a yield analysis application when the system detects that there is sufficient yield via the aggregated biosample yield technologies described herein. The overall job finishes faster.

Numerous other benefits can result, such as increased visibility for sequencing progress, improved management of the sequencing workflow, and the like.

### Example 2—Example System Implementing Multiplexed Biological Sample Aggregation

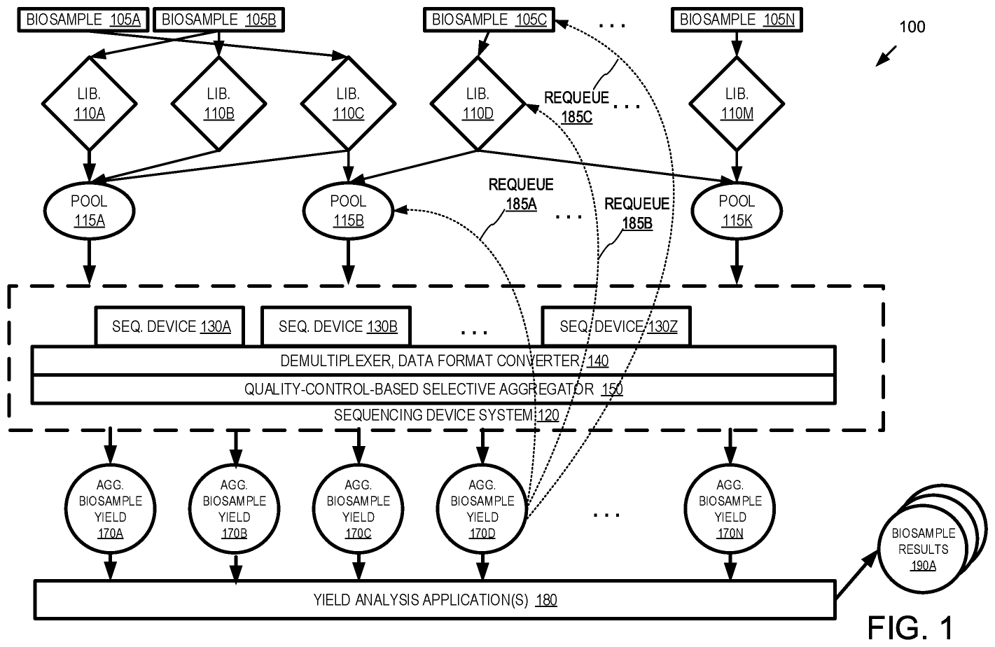

FIG. 1 is a block diagram of an example system 100 implementing multiplexed biological sample aggregation.

In the example, a plurality of biosamples 105A-N are used to prepare related libraries 110A-M. The libraries 110A-M are combined into pools 115A-K. The pools 115A-K are used as physical inputs into a sequencing device system 120. Namely, the pools are sequenced by sequencing devices 130A-Z.

The sequencing devices 130A-Z perform sequencing runs and output raw sequencing data that is demultiplexed and format converted by the demultiplexer, data format converter 140, which outputs sequencing yield data sets to the quality-control-based selective aggregator 150, which can perform the aggregation methods described herein.

As described herein, the quality-control-based selective aggregator 150 can aggregate sequencing yield for respective of the biosamples 105A-N, track yield progress, take quality control metrics into account, and automatically launch a yield analysis application 180 with the aggregated biosample yield 170A-N (e.g., sequencing yield data sets) when sufficient yield is aggregated. Any of the methods related to aggregation described herein can be performed by the aggregator 150.

Although a single yield analysis application 180 is shown, in practice, different applications can be used to analyze yield for different biosamples. And, different applications can also be used to analyze yield for the same biosample.

Although not shown, internal representations of sequencing entities can be stored in one or more computer-readable media. For example, internal representations of sequencing runs, lanes, libraries, biosamples, and the like can be stored as run identifiers, lane identifiers, library identifiers, biosample identifiers, and the like. Relationships between the entities can also be stored to indicate which lanes are related to which runs, and so forth. The yield aggregator 150 can be configured to receive a demultiplexed candidate biosample sequencing yield data set originating from multiplexed raw biosample sequencing data and determine, from the internal representations, that the data set originates from a particular biosample, aggregate the data set with other data sets originating from the same particular biosample, and calculate a total amount of yield acquired for the particular biosample (e.g., by adding the yield from aggregated data sets together).

The application 180 can then produce biosample results 190A-N. Due to the volume of data and the complexity of the analysis, it is not unusual for the yield analysis application 180 to take a significant amount of time (e.g., hours, days, or the like) to complete. Therefore, it is advantageous to begin the analysis soon after a sufficient amount of yield is available (e.g., regardless of the time of day, whether a scientist is presently aware that the yield is available, or whether the laboratory is even staffed at the time).

Still further analysis can be performed on the biosample results 190A-N.

As described herein, it is sometimes desirable for a variety of reasons to request additional yield for a biosample. For example, the aggregated biosample yield actually acquired from an initial sequencing request may not be sufficient. The technologies herein can support requeue requests 185A-C, which can specify additional sequencing is to take place. Depending on quality control and/or remaining physical biological material, such requeues can take place at different levels (e.g., the pool level 185A, the library level, 185B, or the biosample level, 185C). Additional yield can then be sequenced, acquired, and aggregated as described herein.

In any of the examples herein, although some of the subsystems are shown in a single box, in practice, they can be implemented as systems having more than one device. Boundaries between the components can be varied. For example, although the demultiplexer, data format converter 140 is shown as a single entity, it can be implemented by a plurality of devices across a plurality of physical locations.

In practice, the systems shown herein, such as system 100, can vary in complexity, with additional functionality, more complex components, and the like. For example, additional services can be implemented as part of the sequencing devices 130A-Z. Additional components can be included to implement cloud-based computing, security, redundancy, load balancing, auditing, and the like.

In practice, the systems shown herein, such as system 100 can be implemented as part of an automated sequencing orchestration environment that provides a variety of functionality to manage sequencing tasks and subsequent analysis (e.g., an automated workspace within which scientists can achieve their research or experiment goals). Such an environment can implement cloud-based functionality for flexibility and collaborative purposes. While some parts of the system are implemented in a sequencing instrument itself (e.g., the pools 115A-K are analyzed within the devices 130A-Z), other parts of the system can be implemented in the sequencing orchestration environment. The actual division of labor between the sequencing devices and the environment can vary. In practice, the aggregator 150 and yield analysis application 180 are typically part of the sequencing orchestration environment. The demultiplexer, data format converter can be realized within devices 130B or within the environment.

The described system 100 can integrate with a laboratory information management system as described herein.

The described systems can be networked via wired or wireless network connections to a global computer network (e.g., the Internet). Alternatively, systems can be connected through an intranet connection (e.g., in a corporate environment, government environment, educational environment, research environment, or the like).

The system 100 and any of the other systems described herein can be implemented in conjunction with any of the hardware components described herein, such as the computing systems described below (e.g., processing units, memory, and the like). In any of the examples herein, the inputs, outputs, aggregated biosample yield, biosample yield progress, configuration information, and the like can be stored in one or more computer-readable storage media or computer-readable storage devices. The technologies described herein can be generic to the specifics of operating systems or hardware and can be applied in any variety of environments to take advantage of the described features.

### Example 3—Example Method of Multiplexed Biological Sample Aggregation

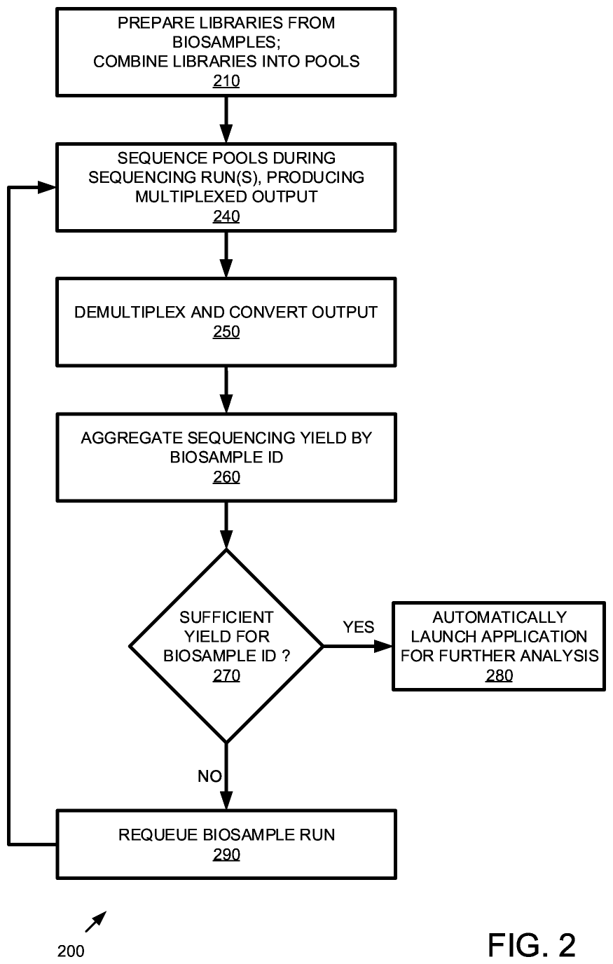

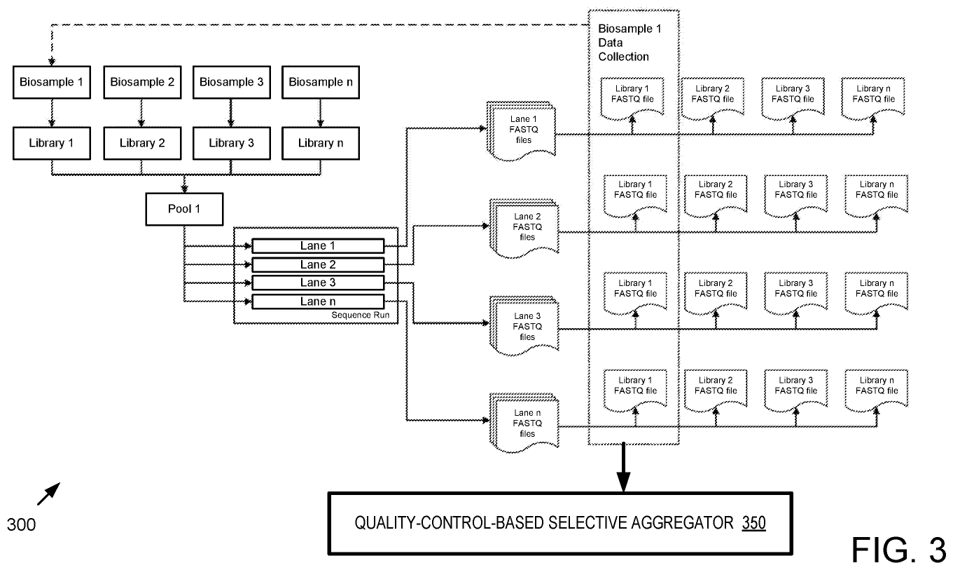

FIG. 2 is a flowchart of an example method 200 of implementing multiplexed biological sample aggregation and can be implemented, for example, in a system such as that shown in FIG. 1. A plurality of biosamples can be supported.

In practice, actions can be taken before the process begins. For example, a scientist may decide to set up a series of experiments involving multiple biosamples. Or, lab personnel may arrange biosample analyses to increase efficiency while maintaining integrity of the process. As described herein, requeue functionality can also be supported to acquire additional yield when insufficient yield is available.

At 210, libraries are prepared from biosamples in a laboratory. In practice, the logistics of such preparation can be organized by preparing and submitting a work order specifying various details of the biosample, a related library (e.g., prep kit), and other related information. The library can be associated with a distinct sequence that allows identification of results for the biosample to be recognized in pooling scenarios. Such an arrangement is sometimes called “barcoding” because the sequence effectively serves as a barcode identifier in sequencing results produced by the sequencing instrument.

If desired, libraries can be combined into pools, resulting in multiplexed sequencing as described herein. However, many of the features herein can be implemented without using pooling. Thus, unmultiplexed aggregation can also be implemented (e.g., to aggregate yield for biosamples where at least one biosample is sequenced via a pool containing a single library in a lane or sequencing instrument). Such unmultiplexed aggregation can still provide many of the benefits described herein.

At 240, the pools are sequenced during one or more sequencing runs, producing multiplexed output. In practice, sequencing runs can be run in parallel so that more than one sequencing run (e.g., on more than one instrument) is performed at the same time. Parallelism can also be achieved in that sequencing takes place over more than one sequencing lane per instrument. A sequencing instrument itself can produce multiplexed output in that sequencing data for more than one biosample (e.g., library associated with the biosample) can be produced during a single sequencing run.

At 250, the output from the sequencing runs are demultiplexed, and the format of the data is converted from a raw data format into a sequencing yield format (e.g., conversion from .bcl files to FASTQ datasets segregated by library). As described herein, in practice, biosamples are associated with one or more libraries, which allow correlation back to the biosample by identification of a barcode associated with the library in the raw data format.

Evaluation of quality control metrics can influence the aggregation process. For example, if some results are identified as having failed quality control, the results can be excluded from aggregation. Thus, quality-control-based-selective aggregation can be implemented. A wide variety of quality control metrics and scenarios can be implemented as described herein, including explicit override of automated quality control failures.

At 260, based on identification of a biosample identifier, sequencing yield is aggregated by biosample. For example, although a set of sequencing runs may involve many different biosamples, the described technologies are able to coordinate aggregation of sequencing yield by biosample across the runs, including in simple scenarios or more complex scenarios involving pooling, parallel sequencing across lanes, parallel sequencing across instruments, requeues due to quality control failures, and the like.

At 270, it is determined whether there is sufficient yield for the biosample (e.g., as identified by the biosample identifier). For example, the associated electronic work order can specify a target number of base pairs as sufficient. The work order can further specify an application to be launched with the yield as input when sufficient yield is acquired. As described herein, the determination of sufficient yield can involve a number of factors, including quality control determinations, yield-in-progress, and other techniques, so that a realistic, accurate determination can be made regarding whether there actually is sufficient usable yield, whether it is advisable to request additional yield, or the like.

Responsive to determining that there is sufficient yield, an application (e.g., specified in the associated work order) is automatically launched at 280 and provided with the aggregated sequencing yield as input.

On the other hand, responsive to determining that there is insufficient yield, an appropriate alert can be generated, resulting in a requeue of the biosample run at 290. The process then results in further sequencing activity. Although the example shows a requeue scenario involving sequencing an existing pool, other requeue scenarios are possible as described herein. The sequencing results of the requeue are then eventually matched and aggregated for the biosample as well, leading to a re-evaluation of whether there is sufficient yield. Multiple requeues are possible.

As part of the requeue process, yield-in-progress can be accounted for. For example, a certain amount of yield can be designated as “pending,” and such yield can be taken into account when determining whether there is sufficient yield as described herein.

### Example 4—Example Biological Sample

In any of the examples herein, a biological sample (or “biosample” or simply “sample”) can be used as a physical input to the technologies. In practice, such a biological sample can take the form of a mass of biological material originating from a living organism. For example, organic tissue from saliva, blood, tumor, or organs can be acquired and processed into a form that is suitable for sequencing or library preparation. In some scenarios, it is desirable to limit the biosample to one particular organism (e.g., an organism having a shared genome), but multi-organism biosamples can be supported.

A biosample preparation request can be a request to sequence a certain amount of data. Such yield is called “target yield” or “required yield” herein. To facilitate tracking of biosamples within the sequencing system, a biosample identifier (or “biosample id”) can be assigned to particular biosamples and stored within various components of the system. For example, a biosample identifier can be associated with a particular library that is sequenced on a particular instrument, lane, or the like. Subsequently, when sequencing data are provided by the sequencing instrument, the data can be matched to the biosample identifier, allowing determination of whether there is sufficient yield as described herein.

Therefore, when the term “biosample” is used herein, it is often synonymous with “biosample identifier.” For example, in practice, determining whether there is sufficient yield for a biosample takes the form of determining whether there is sufficient yield for (a biosample identified by) a biosample identifier. Conversely, when “biosample identifier” or “biosample id” are used, a biosample is indicated.

### Example 5—Example Biosample Manifest

In any of the examples herein, an electronic biosample manifest can be stored that indicates a biosample name, project, container name, container, prep request, target yield (e.g., in Gbp), analysis workflow, sample label, delivery mode, source, and sample type. The manifest can indicate that certain samples are grouped (e.g., to be analyzed together by a yield analysis program). In the case of groups, automatic launching of the application can take place responsive to determining that sufficient yield is acquired for members of the group.

### Example 6—Example Index Sequence

In any of the examples herein, multiplexed sequencing can be accomplished by using an index sequence (or simply “index”). In practice, a biosample preparation kit can prepare the biosample for sequencing by creating a library such that a distinctive sequence of bases is detected for the biosample during sequencing. Other biosamples can have other indexes, so the results can be differentiated even though they sequenced together. The index sequence is sometimes called a “barcode” because it serves as a distinguisher among sequences read during the sequencing process.

As described herein, a single biosample may be sequenced across a plurality of sequencing instruments. In such a case, it is possible for a single biosample to be associated with a plurality of different indexes (e.g., a first index in a first pool being sequenced on a first instrument, a second, different index in a second pool being sequenced on a second instrument, and so forth). Conversely, the same index may be used for more than one biosample (e.g., a first biosample in a first pool being sequenced on a first instrument may use the same index as a second biosample in another pool being sequenced on a second instrument). Thus, although there is some correlation between biosamples and indexes, the biosample identifier is not always matched to the same index identifier; therefore they cannot always be used interchangeably. Other information, such that accumulated from a sample sheet that specifies the biosample identifier can be used as described herein to fully correlate sequencing data with a particular biosample. Quality control and aggregation can then be accomplished as described herein.

Internally, the index sequence can be represented in computer-readable media as a string. For example, valid characters can be A, C, G, and T. “N” can also be included, where “N” matches any base.

An index can have an associated index identifier (e.g., a number of other identifier) assigned by a sequencing orchestration environment for tracking and/or display purposes. Such an identifier is sometimes simply called the “index” for purposes of convenience.

### Example 7—Example Sequencing Yield

The actual sequencing yield to be processed can take the form of detected nucleotide sequences within the biosample (e.g., n-mers) that can then be further analyzed (e.g., by a yield analysis application as described herein) to determine characteristics of the biosample.

In practice, the amount of yield is an important part of the process because a sufficient amount of yield is typically designated as needed to perform further analysis. Therefore, the term “yield” is sometimes used to denote simply an amount of yield. In practice, the amount of yield can be designated by base pairs (bp), giga base pairs (Gbp or Gb), or the like.

### Example 8—Example Yield Aggregation

In any of the examples herein, sequencing yield can be aggregated by biosample. In other words, per-biosample sequencing yield aggregation can be implemented. So, the yield from a variety of different yield paths for a particular biosample can be combined with other yield from that particular biosample, whereas yield from other biosamples are not combined with the yield from the particular biosample. Such a process can be performed for a plurality of biosamples, resulting in aggregated yield for a number biosamples, each segregated by biosample.

In practice, the yield can take the form of aggregating sequencing yield data sets (e.g., FASTQ files) into sequencing yield data yield for a particular data sample. Because such data sets may be rejected as part of the aggregation process (e.g., whether because they are from another biosample, do not meet quality control, or the like), they are sometimes initially called “candidate biosample sequencing yield data sets.” Such candidate data sets that are identified as originating from a particular biosample and also meeting quality control are actually aggregated.

Although the term “combined” is used herein, yield combination can take the form of logical combination. For example, a set of files with yield results can be designated as belonging to the same biosample without actually combining the files together. However, at some point during analysis, combination can be performed as desired.

Selecting which data sets to include based on quality control is sometimes called “selective aggregation” because some data determined not to meet quality control can be excluded from (e.g., not selected for) aggregation. So, in any of the examples herein, aggregation can take the form of quality-control-based selective aggregation in that yield that is detected or designated as failing quality control can be excluded from (e.g., filtered out of) aggregation.

As described herein, sequencing progress per biosample can be monitored by the system by monitoring the number of base pairs of acquired yield, as well as accounting for yield-in-progress, failed yield, and the like.

In this way, a clear and accurate picture of sequencing progress for a particular biosample can be determined, and the sequencing process can be managed to increase efficiency and reduce waste.

### Example 9—Example Sequencing Instrument

In any of the examples herein, a sequencing instrument (also called a “sequencing device” or “device”) can be used to generate sequence data for biosamples. In practice, the sequencing instrument observes nucleotide sequences present in the biosample, and such sequences are typically used in an overall process that is sometimes called “sequencing the biosample.”

The technologies described herein can use any of a variety of sequencing hardware, including the ILLUMINA line of sequencing instruments available from Illumina, Inc. of San Diego, Calif., including the MiniSeq, HiSeq, MiSeq, HiScanSQ, NextSeq, or NovaSeq instruments.

### Example 10—Example Requeue

In any of the examples herein, biosample sequencing can be requeued. In practice, many sequencing tasks may complete without incident, and further analysis of the resulting sequencing data can take place without having to do requeue processing. For example, in scenarios where a yield analysis application is specified along with an indication of sufficient yield, the application can be automatically launched when sufficient yield is acquired.

However, for any of a variety of reasons, there may be a failure in the sequencing process, whether it be a failure in accumulation of data or in confidence of results. Failures can result from one or more quality control metrics (e.g., being outside one or more respective thresholds), a faulty biosample, improper preparation of the biosample, faulty equipment or reagents, interference among components, physical aberrations, or any of a number of other variables.

In such a situation, it is typically desired to restart some stage of the sequencing process and acquire additional yield so that sufficient yield can eventually be acquired.

As described herein, when it is discovered that there is insufficient yield, a requeue alert can be raised. In practice, a missing yield alert can serve as a requeue alert.

The user interface associated with the missing yield condition can facilitate easy launch of a requeue, and the requeue process can include accounting for yield-in-progress associated with the requeue as well as preparing to match the yield to the request when the yield from the requeue arrives.

As described herein, the requeue can take place at different stages of the sequencing process depending on where failure occurred and/or how much physical material remains to be sequenced. For example, if a pool associated with failed yield is available, the pool can simply be resequenced. In some cases, a library other than the one associated with the particular biosample being requeued may be associated, but the decision to requeue can take such a situation into account.

If a remaining quantity of the pool is not available or desired to be sequenced, the prepared library can be resequenced (e.g., whether or not combined into a pool). And, if a remaining quantity of the library is not available or desired to be sequenced, the biosample itself can be used to prepare more or a different library material for sequencing. Library types can similarly be involved.

The work order associated with the requeue can be associated with the biosample, and the work order can be designated as a requeue. So, when the yield is eventually provided, it can be matched to the requeue request as described herein. The yield can then be aggregated to other yield for the biosample, and the progress (e.g., pending yield or the like) can be updated for further determination if there is sufficient yield.

### Example 11—Example Missing Yield Condition Alert

In any of the examples herein, a missing yield condition alert (or “missing yield alert”) can take the form of an explicit message, a display of yield that shows yield is missing, or the like.

For example, an alert can be raised, displayed, or communicated to prompt action by a user. Or, during display of progress on a dashboard for biosamples being sequenced, the amount of yield for respective biosamples can indicate progress. Missing yield can be indicated on the dashboard (e.g., implied or explicitly by displaying yield for those biosamples having missing yield in a distinctive color, or the like).

In practice, a missing yield condition alert can serve as a requeue alert. The user interface associated with the missing yield condition can facilitate easy launch of a requeue (e.g., the appropriate work order, designated as a requeue work order). Thus, the missing yield alert can comprise a user interface element for requesting a requeue of sequencing processing for the particular biosample. For example, a graphical button can be displayed, and responsive to activation of the button, a workflow for the requeue can be started, including collecting information for a work order or information that is eventually included in such a work order. The information can be stored and subsequently matched with incoming yield datasets so that aggregation can be achieved. Such information can include the biosample identifier, library, instrument, lane information, amount of expected yield, or the like.

### Example 12—Example Work Order

In any of the examples herein, work orders can take a variety of electronic forms. In practice, the work order can be an indication that directs sequencing activity and is stored and communicated by the sequencing system electronically. For example, a work order can request preparation and sequencing of a biosample. So, the work order can contain or take the form of a preparation request (or “prep request”) specifying that a biosample be prepared and sequenced. An electronic sample sheet can contain further information that facilitates sequencing activity, and the work order can reference (e.g., link to) the sample sheet.

The work order can specify how the biosample is to be prepared (e.g., the type of kit used to prepare the library or the like).

As described herein, the work order can further specify what is sufficient sequencing yield and an application to be launched upon acquisition of such sequencing yield.

Work orders can also be specified for requeues as described herein.

### Example 13—Example Sequencing Orchestration Environment

Any of the examples herein can be implemented in a sequencing orchestration environment. Such an environment can take the form of an automated workspace within which users can monitor, control, and analyze sequencing tasks. A rich set of functionality can also track sample and library preparation, and serve as a center for a variety of sequencing information.

Cloud-based functionality can support connectivity from a variety of locations and devices so that users are able to orchestrate a wide variety of tasks on an ongoing basis.

Relationships between biosample identifiers, electronic sample sheets, multiplexed raw biosample sequencing data output from sequencing instruments, sequencing yield data sets.

### Example 14—Example Yield Analysis Application

In any of the examples herein, a yield analysis application can be executed within a sequencing orchestration environment as described herein. Such applications can be used in the field of genetic analysis, data handling, data quality control, data visualization, gene expression and regulation, microbial genomics, metagenomics, proteomics, and the like. Examples of such applications include those that perform gene expression profiling, exome sequencing, whole-genome sequencing, tumor analysis, forensic analysis, de novo sequencing, and the like.

Such a yield analysis application can perform a variety of functions, such as alignment, variant calling, variant analysis, de novo assembly, phylogenetic analysis, viral typing, pathway analysis, and the like.

Yield analysis applications can be provided by parties other than those providing the underlying sequencing instruments or other components of the sequencing device system. Such applications can be executed in a sequencing orchestration environment and be provided with acquired sequencing yield as described herein.

Implemented examples of such applications include the Amplicon DS application, TruSight Tumor applications, the Tumor Normal application, the Whole Genome Sequencing application, the MethylKit application, and any of a variety of others now available or hereafter developed.

### Example 15—Example Sequencing Entities

In any of the examples herein, various processes can be performed for or across various sequencing entities. Such sequencing entities can include biosample, library, library type, pool, sequencing instrument, sequencing run, flowcell lane, tile, and the like)

### Example 16—Example System Performing a Single Sequencing Run

FIG. 3 is a block diagram of an example system 300 performing a single sequencing run for use in multiplexed biological sample aggregation. In the example, a plurality of biosamples prepared for sequencing, and corresponding libraries are prepared. The biosample to library relationship can be one to many. In other words, a same, single biosample can be used to create one or more libraries.

In practice, more complex scenarios can be implemented. For example, many sequencing runs can be performed in parallel across a plurality of sequencing instruments.

Although not required for sequencing or aggregation, multiple libraries are combined into a single physical pool as shown, which is then analyzed in a single sequencing run that has a number of sequencing lanes that perform sequencing in parallel.

As shown, a single sequencing instrument can analyze a plurality of lanes during a single sequencing run. Analysis of the sequencing lanes produces respective sets of FASTQ files that represent demultiplexed sequencing data (i.e., representing sequencing yield). As this point, the yield is not yet considered acquired because it may suffer from quality control problems. The yield is also not yet considered aggregated because it has not yet been combined into other yield data sets for the same biosample.

As shown, aggregation for a particular biosample (e.g., biosample 1) can be achieved by a quality-control-based selective aggregator 350 by identifying and combining (e.g., associating together) FASTQ files for the particular biosample.

As described herein, sequencing yield progress can be monitored, and eventually the acquired yield for the biosample can be further analyzed.

### Example 17—Example Method Performing a Single Sequencing Run

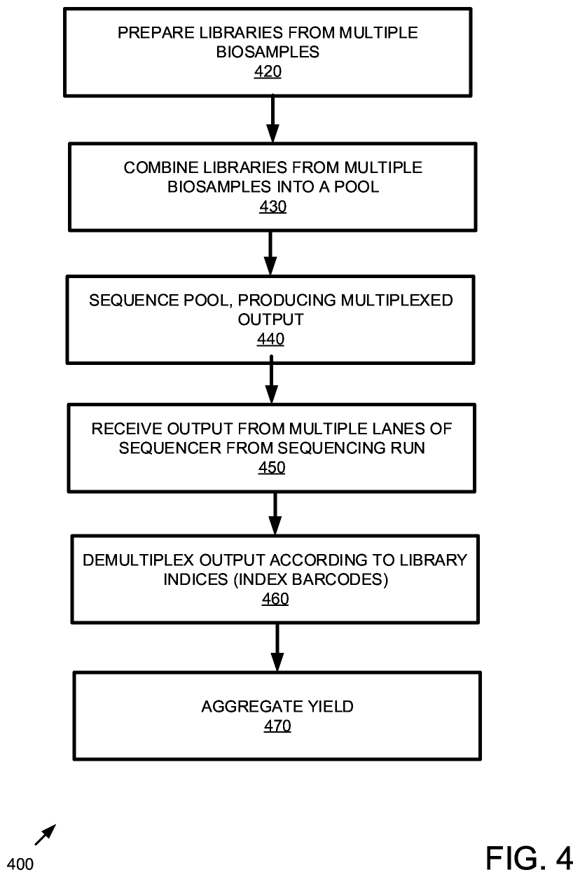

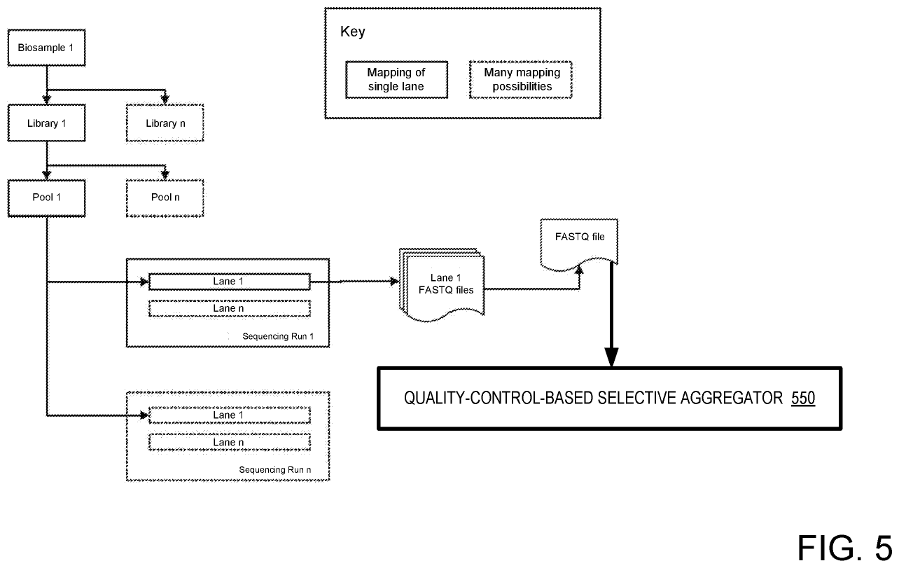

FIG. 4 is flowchart of an example method 400 performing a single sequencing run for use in multiplexed biological sample aggregation and can be performed, for example, by the system of FIG. 3. In practice, the method 400 can be implemented in parallel (e.g., a plurality of sequencing runs are performed on sequencing instruments at the same time).

At 420, libraries for multiple biosamples are prepared as described herein.

Although not required for sequencing or aggregation, at 430, libraries from multiple biosamples are combined into a pool as described herein.

At 440, a sequencing instrument sequences the pool, producing multiplexed output. As described herein, the instrument can have multiple lanes.

At 450, the output from multiple lanes of the sequencer are received from the sequencing run.

At 460, output can be demultiplexed according to the library indexes. For example, different results associated with different libraries are grouped together by library index (e.g., index barcode).

At 470, the yield for a particular is aggregated as described herein. In practice, identification of the library correlates with a biosample. The incoming yield from the sequencing instrument can be matched to a particular biosample (e.g., via association of the biosample with a work order, library, or the like).

### Example 18—Example Library Types

Although many of the descriptions herein refer to libraries in a generic sense, it is possible to have different library types. In any of the examples describing aggregation, aggregation by library type is also possible.

A wide variety of library prep kit types can be used for the preparation of different library types from a biosample. A biosample can be used to generate one or more libraries of a particular type, and the aggregation of sequencing data for a biosample can be performed distinctly against each library type. For example, Biosample 1 can be used to generate libraries of type A (say instance A1, A2, and A3) and type B (say instance B1 and B2), and when sequencing data is aggregated for Biosample 1, data from A1, A2, and A3 are aggregated separately from data from B1 and B2.

For example,
Biosample 1
   Library (type A)—aggregated together (A1+A2+A3)
   Library (type b)—aggregated together (B1+B2)

In this way, analyses can specify that a certain amount of yield for different library types is sufficient (e.g., 40 Gbp of type A and 20 Gbp of type B). Requeue and progress functionality can be extended to library types (e.g., an alert specifies that more yield of library type A is needed and a requeue is implemented and eventually aggregated back as yield for the biosample as yield of library type A).

### Example 19—Example System Performing a Single Sequencing Run

FIG. 5 is a block diagram of example relationships 500 for sequencing entities in multiplexed biological sample aggregation scenarios. In practice, such relationships can become complex and burdensome to track and analyze. The technologies described herein can free scientists and other users from having to concern themselves with such complexities and focus on the ultimate goal of their research or experiment.

In practice, a single biosample can be processed into one or more libraries, and such libraries can be of different types as described herein.

A particular library can find its way into one or more pool (and, a pool can contain one more libraries).

The pool can then be sequenced in one or more sequencing lanes in one or more sequencing runs (e.g., performed by one or more sequencing instruments).

Sequencing results of the run for a single sequencing lane can result in one or more sequencing yield data sets (e.g., FASTQ files), and any sequencing yield data set can be used as input to a quality-control-based selective aggregator 550 to implement aggregation as described herein.

### Example 20—Example Method Performing a Single Sequencing Run

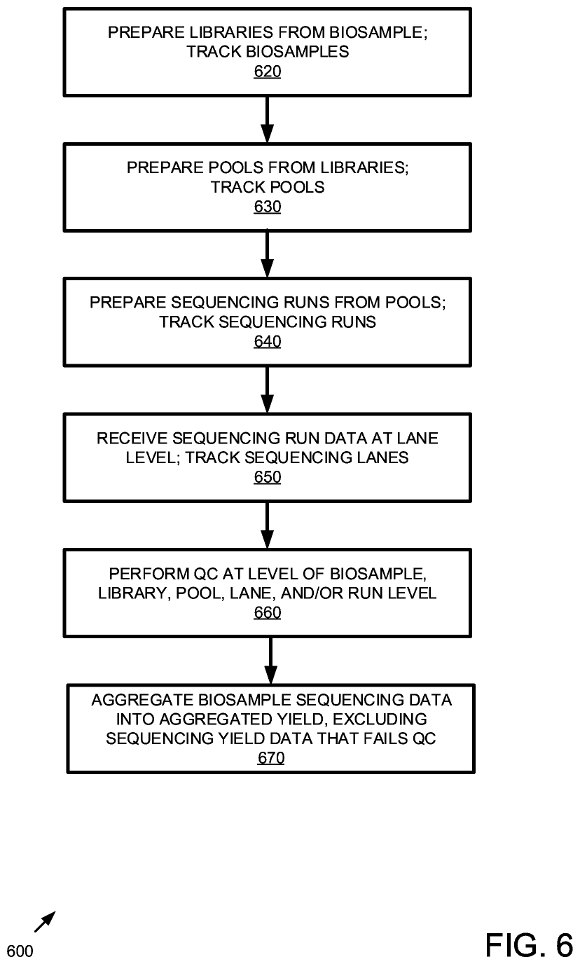

FIG. 6 is flowchart of an example method 600 processing sequencing entities in multiplexed biological sample aggregation scenarios and can be implemented, for example, according to the arrangement of FIG. 5.

At 620, one or more libraries are prepared from a biosample. Biosamples are tracked so that relationships between libraries (e.g., that are identified by a distinctive nucleotide string) are stored and can be used to correlate sequencing results to a particular biosample for aggregation purposes.

At 630, one or more pools are prepared from a biosample. Pools can also be tracked. For example, pools can be associated with particular lanes of particular sequencing runs.

At 640, one or more sequencing runs with one or more lanes are prepared, and such sequencing runs can be tracked for purposes of later aggregating the yield to the biosample.

At 650, raw biosample sequencing data for the biosamples of the sequencing run are received. In practice, the data is received at the lane level, and sequencing lanes can be tracked as described herein. Demultiplexing can convert the raw data into sequencing yield data sets.

At 660, quality control can be performed at the level of biosample, library, pool, lane, and/or run level. As described herein, automated quality control metrics can be implemented, and a user can override such automated determinations.

At 670, biosample sequencing yield data sets for a particular biosample are aggregated into aggregated yield, excluding sequencing yield data that does not meet quality control as described herein.

### Example 21—Example System Performing Aggregation Across Sequencing Entities

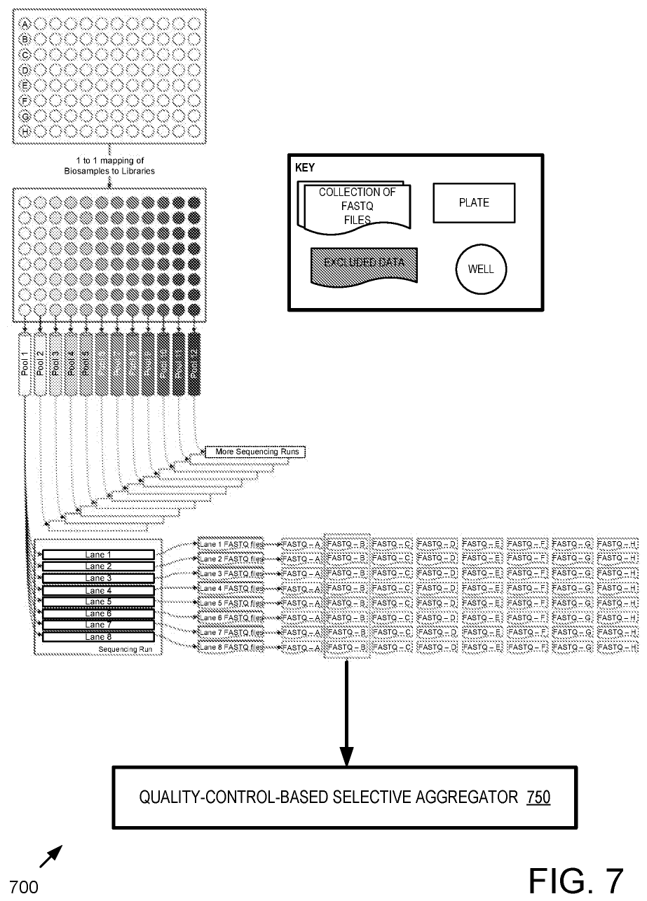

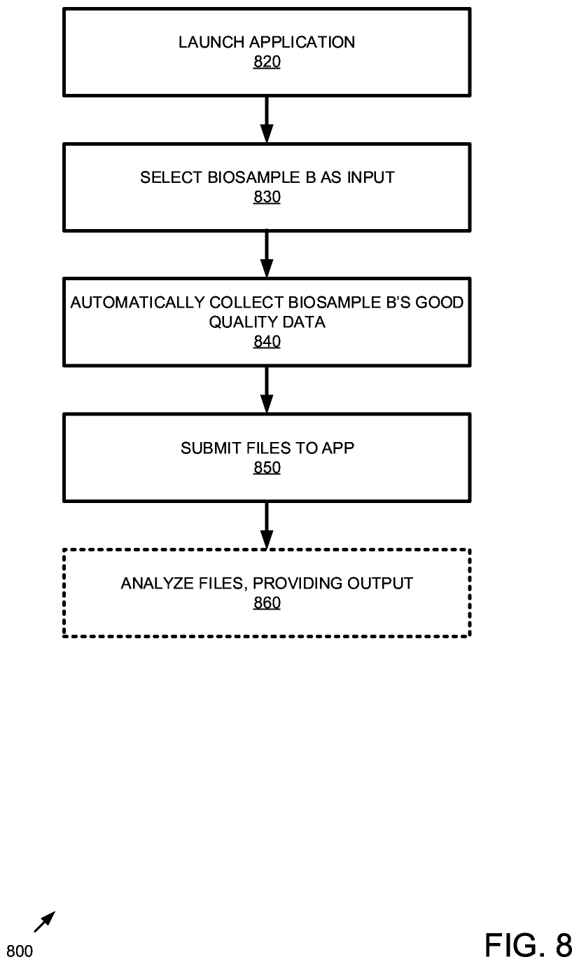

FIG. 7 is a block diagram of an example system 500 aggregating yield from multiplexed biological samples. In the example, there is a 1:1 mapping of biosamples to libraries, and biosamples A-H are analyzed in parallel.

Multiple libraries are combined into pools 1-12, which are analyzed by a plurality of sequencing runs.

A particular sequencing run with 8 lanes is shown for illustration. For the sequencing run, the raw data are demultiplexed into are 8 groups of biosample sequencing yield data sets (i.e., one for each lane). The yield data sets can be grouped by related sample, even though the data comes from different lanes.

A quality-control-based-selective aggregator 750 can receive the biosample sequencing yield data sets and aggregate the yield for a particular biosample that meets quality control as described herein. Although the drawing shows aggregation for a single sequencing run, in practice, aggregation can aggregate across sequencing runs.

### Example 22—Example Method Performing Aggregation Across Sequencing Entities

FIG. 8 is flowchart of an example method 800 of aggregating yield from multiplexed biological samples and can be implemented, for example, in the arrangement shown of FIG. 7. At 820, a yield analysis application is launched for analysis of yield. At 830, biosample B is selected as input. In practice, a biosample identifier or name can be provided.

At 840 Biosample B's good quality data (e.g., the biosample sequencing yield data sets) meeting quality control are collected, resulting in aggregation.

At 850, the good quality data files are submitted to the application.

At 860, the files are analyzed, and the application provides output.

### Example 23—Example Modalities of Aggregation

In any of the examples herein, various modalities can be used for aggregation. One example modality is to aggregate on demand, in response to a request. FIG. 8 shows such a scenario. Yield data arrives and is stored. A user can activate a yield analysis application (e.g., by selecting a button in a user interface). Aggregation can then take place, and the aggregated data is used as input by the yield analysis application.

Alternatively, aggregation can be performed on an ongoing basis. For example, events indicating arrival of incoming yield (e.g., biosample sequencing yield data sets) can be detected, and the incoming yield can be aggregated. As part of setting up biosample workflow, the requesting user can specify a particular yield analysis application to be launched in response to acquiring a specified amount of yield. The user need not take further action after specifying (e.g., assuming the yield is acquired). As described herein, an application can be launched when sufficient yield is acquired.

### Example 24—Example System Performing Selective Aggregation Across Sequencing Entities

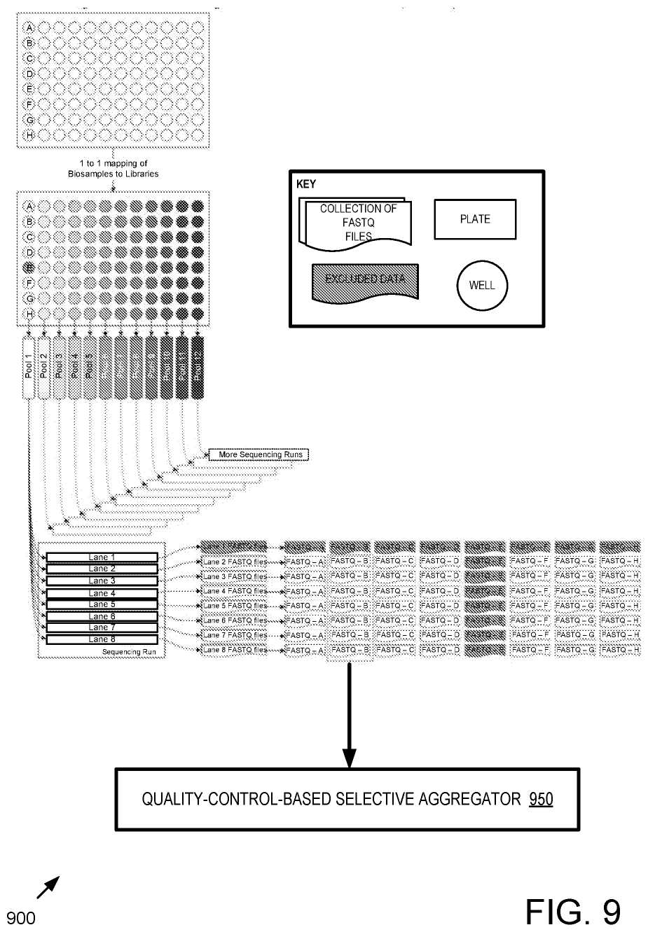

FIG. 9 is a block diagram of an example system 900 selectively aggregating yield from multiplexed biological samples. The scenario parallels that of FIG. 7. However, it has been determined that a particular lane (i.e., lane 1) and a particular library (i.e., library E) have failed quality control. As a result, the sequencing yield data sets for such entities are not included in aggregation by the quality-control-based selective aggregator 950.

Although failure is shown at the lane and library level, quality control can be used to detect failure at the level of any of the various sequencing entities described herein.

### Example 25—Example Raw Biosample Sequencing Data

In any of the examples described herein, raw biosample sequencing data can contain sequences read for a plurality of biosamples being simultaneously sequenced by a single instrument. Therefore, the raw output contains observations of actual base sequences (e.g., n-mers) present in physical biosamples and typically takes the form of multiplexed data. In practice, a plurality of such instruments can be performing sequencing in parallel.

An example of such data are .bcl files generated by the ILLUMINA line of sequencing instruments available from Illumina, Inc. of San Diego, Calif., and can be named to include the lane and tile involved. Such files can encode bases that are read by the instrument in a code (e.g., using 0, 1, 2, 3 for A, C, G, T or the like). However, other formats can be used to generate yield datasets that can be aggregated as described herein.

Such raw data is often of little use in its raw form because while it does indicate sequences read by the instrument, the actual sequences of a particular sample are intermingled with those of other biosamples.

In practice, such data can be demultiplexed and converted into a form more usable for various purposes as described herein (e.g., by a demultiplexer, data format converter as described herein). Further, although demultiplexed scenarios are described herein, the technologies can still be applied to scenarios where there is at least some data that is not multiplexed (e.g., the output is for a single biosample that is analyzed by a single instrument, and there are a plurality of such instruments operating in parallel).

### Example 26—Example Sequencing Yield Dataset

In any of the examples herein, a sequencing yield data set can include the data converted and demultiplexed from raw biosample sequencing data originating from the sequencing instrument. A demultiplexer, data format converter can accept the raw biosample sequencing data and output a plurality of sequencing yield datasets for respective libraries.

In practice, a single yield data set is associated with a particular biosample, or in practice a single library, which is then associated with a particular biosample. A sequencing yield dataset can indicate the barcode sequence of the library read during sequencing so that the barcode can be correlated with a biosample. For example, the barcode (e.g., index identifier) can be incorporated into the file name or otherwise stored as associated with the dataset.

An example of such datasets are FASTQ files that store both a nucleotide sequence and corresponding quality scores. Such FASTQ files can be generated by the ILLUMINA sequencing device systems and are used to store the output of sequencing instruments in a useful form.

Besides indicating the actual sequence itself, the dataset can include further information as desired, such as the instrument identifier, run number on the instrument, flowcell identifier, lane, tile, quality information, and the like.

In practice, a plurality of such yield datasets are generated from a single sequencing run, and the datasets can then be aggregated as described herein. As described herein, the determination of whether there is sufficient yield can be based on whether there is sufficient yield indicated in aggregated yield datasets (e.g., based on the number of base pairs indicated by the combined total length of sequences observed as indicated in the sequencing yield datasets).

### Example 27—Example Demultiplexing and Conversion

In any of the examples herein, a demultiplexer, data format converter (e.g., 140) can accept raw biosample sequencing data (e.g., a file output by a sequencer such as a .bcl file), read the lines of data, identify libraries referred to therein, aggregate the data for a particular library, and output a sequencing yield data set (e.g., one or more FASTQ files) for each library represented in the raw data. Sequencing yield data files can be granular at the run, lane, or other level (e.g., the data for a particular lane is included in one FASTQ file, and data for another lane is included in a different FASTQ file), resulting in multiple files per library. Data is also converted to FAST format, which can include quality information for the sequences that have been read by the instrument.

As described herein, library information can then be used to correlate to a particular biosample and identify which sequencing yield data set is associated with which biosample.

### Example 28—Example Implementation of Quality Control into Aggregation

In any of the examples herein, automated quality control can be incorporated into the aggregation process. So, for example, a portion of the biosample sequencing data can be identified as failing a quality control metric, and responsive to determining that the portion of data failed the quality control metric, the portion can be excluded from aggregation. For example, a portion of candidate biosample sequencing yield data sets can be identified as failing a quality control metric, and responsive to such a determination, the portion of data sets can be excluded from aggregation. Such a portion can comprise one or more data sets.

As described herein, identifying a portion of the biosample sequencing data as failing a quality control metric can comprise comparing an observed quality control metric value (e.g., for the portion, a particular data set, or the like) to a stored threshold value for the quality control metric. For example, for a particular sequencing run performed by a particular sequencing device, a sequencing lane can be identified as failing the quality control metric. Any biosample sequencing data (e.g., data sets) for the failing lane (e.g., and the involved run) can then be excluded from aggregation. Data from a plurality of biosamples (e.g., the particular biosample and other biosamples sequenced in the lane) can be excluded.

As described herein, further responsive to determining that the portion of data failed quality control, a yield status can be updated for the particular biosample to indicate that the excluded yield failed.

After a portion of the biosample sequencing data is excluded from aggregation, an indication to requeue a request for yield for the particular biosample can be received. The request for yield can be requeued, and a yield status can be updated to reflect the requeued request for yield as described herein. A request for yield status can then indicate both acquired yield and a yield-in-progress for the particular biosample. Yield expected from the requeued request can be included for yield in calculations for determining whether enough yield has been requested for the particular biosample. Yield expected from in-progress demultiplexing or format conversion can be included in such calculations.

As described herein, such automated determinations can be overridden. So, after the portion is identified as failing a quality control metric, the portion can be indicated as failed. Then, via user input, an override of the determination can be received. Responsive to receiving the override, the portion can then be included in aggregation.

Although examples show failure at the biosample sequencing yield data set level, failure can also be detected at other levels, such as at the raw data level, aggregated data level, or analysis level.

### Example 29—Example Method of Implementing Quality-Control-Based Selective Aggregation

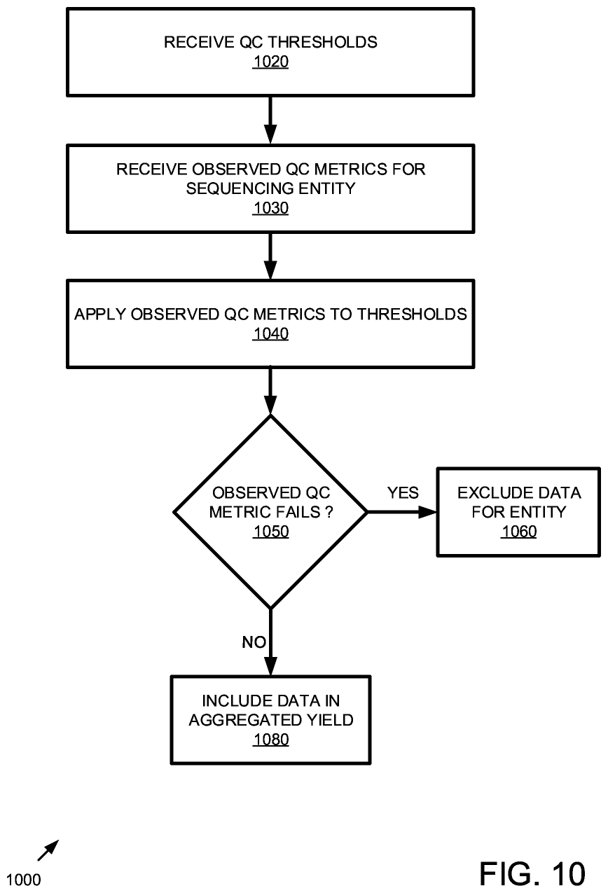

FIG. 10 is flowchart of an example method 1000 of implementing quality-control-based selective aggregation and can be implemented in any of the aggregation examples described herein.

At 1020, quality control thresholds for quality control metrics are received. The system can support any of a wide variety of quality control metrics received during different phases of the sequencing process and subsequent analysis. Thresholds for such metrics can be specified in terms of simple thresholds, combined thresholds, rules, and the like.

In practice, different labs, different users, different experiments, different biosample types, and the like can have different thresholds specified. So, thresholds (and which metrics to consider) can be configured separately in a system per user.

At 1030, observed quality control metrics are received for a sequencing entity, whether from analysis directly associated with the entity or downstream analysis. Such metrics can be included in raw sequencing data, biosample sequencing yield data sets, or downstream analysis. Although examples are shown of lane quality control failures, quality control failures can be implemented at different stages and entities of the sequencing process as described herein (e.g., biosample, library, library type, pool, run, and the like).

At 1040, the observed quality control metrics are applied to the thresholds. For example, a comparison between an observed value and a threshold value can be made for one or more quality control metrics.

It is then determined whether an observed quality control metric meets or fails the thresholds. Responsive to determining that the metric fails, data (e.g., biosample sequencing yield data sets) for the associated entity are excluded from aggregation at 1060. Databases tracking such data can be updated to indicate a failure and why the failure occurred (e.g., the metric, rule, or the like that caused failure).

Conversely, if the quality control metric meets the threshold, the data is included in aggregated yield at 1080.

The yield determinations 1060, 1080 can be implemented on an automated basis so that automatic comparison of quality control metrics occurs (e.g., upon completion of a run, completion of an analysis, or the like). However, a user can override such determinations if desired. For example, if a metric technically fails a metric, but a user determines that such data is still of suitable quality, the designation that such data has failed can be changed to indicate that the data has met quality control, and the resulting yield is then included in aggregation (e.g., and subsequent determination of whether there is sufficient yield).

User interfaces can be employed to help communicate and understand the quality control. So, automatic quality control can compare against the thresholds and tell a user that yield failed and why it failed. Such a user interface can show names of metrics, their thresholds, and observed values (e.g., for a sequencing run).

An example of metrics acquisition is by monitoring data output from sequencing instruments (e.g., parsed from interops), and the like.

### Example 30—Example Downstream Quality Control Failures

In any of the examples herein, it is possible to supplement initial automated quality control with additional downstream quality control failures. For example, it may be determined during analysis of aggregated sequencing yield data sets that there was a quality control failure by some sequencing entity (e.g., lane of the like as described herein). Quality control metrics similar to those associated with the FASTQ files can be applied to yield analysis application output. Failure can indicate some of the upstream data was of low quality. Manual experimentation may also indicate quality control failure (e.g., turning off a lane significantly affects the output).

Even at such late stage, the system can accept an indication that the sequencing entity has failed quality control, and the aggregation results can be updated (e.g., newly failed data is excluded). As a result, the system may now indicate that there is insufficient yield for one or more biosamples, and a requeue process can begin. However, other yield can remain in the system. If desired, failed quality control indication can cascade to yield from the same or other biosamples.

After results of requeued sequencing are then aggregated to existing yield, if there is sufficient yield, analysis can then again be automatically launched or otherwise processed.

An indication of quality control failure for a sequencing entity can thus be received from a user or other source, and the sequencing yield data associated with the indicated sequencing entity can be retrospectively excluded from aggregation, and additional sequencing can then be initiated and tracked until sufficient acquired yield meeting quality control is again indicated.

### Example 31—Example Quality-Control Metrics for Selective Aggregation

For purposes of quality control, a user can select the metrics that are of concern, and the user can set thresholds for such metrics. A sequencing run typically has dozens of metrics that the user can choose for thresholding.

For example, a threshold can specify that a first metrics must be greater than a particular value, and a second metrics must be less than some other value, and so forth.

### Example 32—Example Quality-Control Metrics

Any of a wide variety of metrics can be used for quality control. Metrics can be hierarchically organized into groups for ease of reference. Example metrics that can be used in any of the examples herein follows. Additional or other metrics can be used instead:

### Lane.Density

### Lane.ErrorRate

### Lane.PercentAligned

### Lane.PercentGtQ30

### Lane.PercentPf

### Lane.Phasing

### Lane.PrePhasing

### Lane.Reads

### Lane.ReadsPf

### SequencingRead1.Density

### SequencingRead1.ErrorRate

### SequencingRead1.PercentAligned

### SequencingRead1.PercentGtQ30

### SequencingRead1.PercentPf

### SequencingRead1.Phasing

### SequencingRead1.PrePhasing

### SequencingRead1.Reads

### SequencingRead1.ReadsPf

In practice, failing a quality control metric can involve failing a quality control condition, where such a condition involves one or more metrics and one or more respective thresholds. When a metric is outside of its specified threshold, failure is indicated.

### Example 33—Example Quality-Control Threshold Specification

The following JSON text indicates a set of quality control thresholds according to an acceptable format. In practice, other formats can be used.

```
{
  ″QcThresholds″: [
    {
```

| | |
|---|---|
| ″Name″: ″ErrorRate″, | //Specifying that SR1.ErrorRate < Val |

```
      ″Operator″: ″LessThan″,
      ″ThresholdValues″: [
        <VALUE1 HERE>
      ],
      ″Group″: ″SequencingRead1″
    },
    {
```

| | |
|---|---|
| ″Name″: ″Density″, | //Specifying that lane.density > Val |

```
      ″Operator″: ″GreaterThan″,
      ″ThresholdValues″: [
        <VALUE2 HERE>
      ],
      ″Group″: ″Lane″
    },
    {
```

| | |
|---|---|
| ″Name″: ″PercentGtQ30″, | //Specifying that Lane.%GtQ30 > Val |

```
      ″Operator″: ″GreaterThan″,
      ″ThresholdValues″: [
        <VALUE3 HERE>
      ],
      ″Group″: ″Lane″
    },
    {
      ″Name″: ″PrePhasing″,
      ″Operator″: ″Between″,
      ″ThresholdValues″: [
        <VALUE4 HERE>,
        <VALUE5 HERE>
      ],
      ″Group″: ″SequencingRead2″
    },
    {
```

| | |
|---|---|
| ″Name″: ″Yield″, | //Specifying that lane yield > Val |

```
      ″Operator″: ″GreaterThan″,
      ″ThresholdValues″: [
        <VALUE6 HERE>
      ],
      ″Group″: ″Lane″
    },
    {
```

| | |
|---|---|
| ″Name″: ″Yield″, | //Specifying that SR1.yield > Val |

```
      ″Operator″: ″GreaterThan″,
      ″ThresholdValues″: [
        <VALUE7 HERE>
      ],
      ″Group″: ″SequencingRead1″
  }
]
```

Example results of the thresholds applied to a lane are shown as follows:

| | Observed | | | |
|---|---|---|---|---|
| Metric | Value | Operator | Threshold(s) | Status |
| Lane.Density | 854000 | > | 900000 | NOT met |
| Lane.PercentGtQ30 | 90 | > | 75 | Passed |
| Lane.Phasing | 0.160 | < | 0.5 | Passed |

### Example 34—Example System Identifying Data as Originating from a Particular Biosample

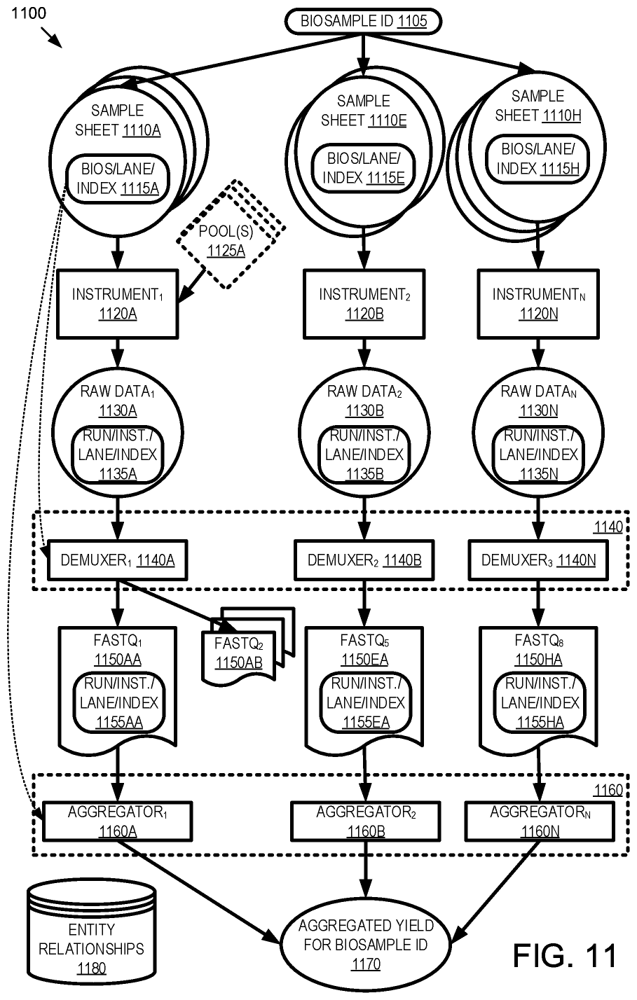

FIG. 11 is a block diagram of an example aggregation system 1100 showing details of how data relating to a particular biosample is identified as originating from a particular biosample, which can be used in any of the examples herein. The example is shown from the perspective of a particular biosample identified by the biosample identifier 1105. In practice, a plurality of biosamples can be processed in parallel, thus leading to the problem of determining which data originates from which biosample. The system 1100 is an example only. Different implementations are possible and can be of greater complexity (e.g., more instruments or the like). Other implementations may appear less complex in some aspects (e.g., components are combined or reused as appropriate). A sequencing orchestration environment can incorporate the system 1100 as described herein.

In the example, the biosample is being sequenced on three different instruments (e.g., in parallel). The sample sheets 1110A, 1110E, and 1110H have information 1110A, 1110E, and 1110H that refer to the same biosample identifier 1105. Other information about which lane of the instrument and an index identifier can also be included in the information 1110A, 1110E, and 1110H as shown. The sample sheets 1110A, 1110E, and 1110H can be used as input to respective sequencing instruments 1120A, 1120B, and 1120N, which sequence the pools 1125A. In practice, sequencing of the biosample associated with the biosample identifier 1105 can be done in parallel with sequencing for other biosamples, which can have their own sample sheets that are shown in the drawing but not labeled.

In practice, the information in the sample sheets 1110A, 1110E, and 1110H can be converted to a format suitable for consumption by the sequencing instruments 1120A, 1120B, and 1120N and sent to instrument control and analysis software. An association (e.g., sample-sheet-identifier-to-instrument-identifier relationship) can be stored (e.g., in entity relationships 1180) between a particular sample sheet 1110A and the associated instrument 1120A based on having passed the data from the sample sheet 1110A to the instrument 1120A. Other ways can be used to associate the information 1115A from the sample sheet 1110A with the instrument 1120A for later correlation. For example, a direct relationship can be stored between the instrument and the information without regard to a sample sheet.

The sequencing instruments 1120A-N output respective multiplexed raw biosample sequencing data 1130A-N for the biosample identified by the biosample identifier 1105 along with other biosamples. The raw data 1130A-N can also include a run identifier identifying the sequencing run (e.g., to identify which sequencing run out of plurality of runs per instrument or across instruments), an instrument identifier (e.g., to identify from which physical instrument 1130A-N the data originates), a lane identifier, and an index identifier as described herein.

The demultiplexer, data format converters 1140A-N can demultiplex the raw data 1130A-N according to index identifier, outputting a plurality of sequencing yield data sets 1150AA-1150HA. Although a plurality of demultiplexers 1140A-N are shown, in practice one or more demultiplexers 1140 can be employed for demultiplexing and conversion.

The sequencing yield data sets 1150AA-1150HA can include information 1155AA-1155HA, comprising a run identifier, instrument identifier, lane identifier, and index identifier. As described herein, the sequencing yield data sets 1150AA-1150HA can be organized by index (e.g., each file has information for one index identifier only).

The data sets 1150AA-1150HA can be treated as candidate biosample sequencing yield data sets. Information identifying the originating biosample may or may not be present in the data sets 1150AA-1150HA. An aggregator 1160A-N can identify which of the data sets 1150AA-1150HA originates from the particular biosample (e.g., identified by the biosample identifier 1105). For example, the aggregator can accept the biosample identifier, lane, and index information 1115A, and use it to correlate between the index identifier in the data sets 1150AA-1150AD and the index identifier from the information 1115A from the sample sheet 1110A (e.g., match the two). Thus, the information 1115 allows the aggregators 1160A-N to differentiate between data sets from different biosamples. In practice, matching index information (e.g., index sequence) may not be sufficient because the same index sequence may be used across different biosamples. Therefore, further information such as a run identifier, instrument identifier, lane identifier, and the like can be used to conclusively match incoming data sets to their respective originating biosamples.

In practice, the information 1115 and additional information can be stored as entity relationships 1180, which can be read by components of the system 1100. For example, relationships between a sample sheet 1110A and the referenced biosample identifier 1105, along with an index identifier, instrument identifier, lane identifier, and the like can represented in rows (e.g., of a database table) or otherwise indicated.

In practice, some information may be implied. For example, information can be stored in a file name or be implied by virtue of its source (e.g., information coming from a particular sequencing instrument can be associated with the instrument identifier of the sequencing instrument, allowing further correlation).

The demultiplexing layer 1140 can also be biosample-aware by consulting the information 11115A-H, entity relationships 1180, or both, and information regarding the origin of the raw data can be used for quality control purposes as described herein.

Although a plurality of aggregators 1160A-N are shown, in practice, one or more aggregators 1160 can be used to accomplish aggregation.

Those data sets identified as originating from the biosample are output (e.g., aggregated) by the aggregators 1160A-N as aggregated sequencing data yield 1170 for the particular biosample identifier by the biosample identifier 1105 (e.g., based on stored entity relationships 1180). As described herein, such output can take the form of the actual sequences read, the number of basepairs involved, or both. In practice, such output can be by reference (e.g., to the data sets 1150AA, 1155EA, 1155HA).

Quality control and requeue functionality can be implemented as described herein, along with sequencing yield progress monitoring and automatic launching of an application when sufficient yield is aggregated.

### Example 35—Example Method Identifying Data as Originating from a Particular Biosample

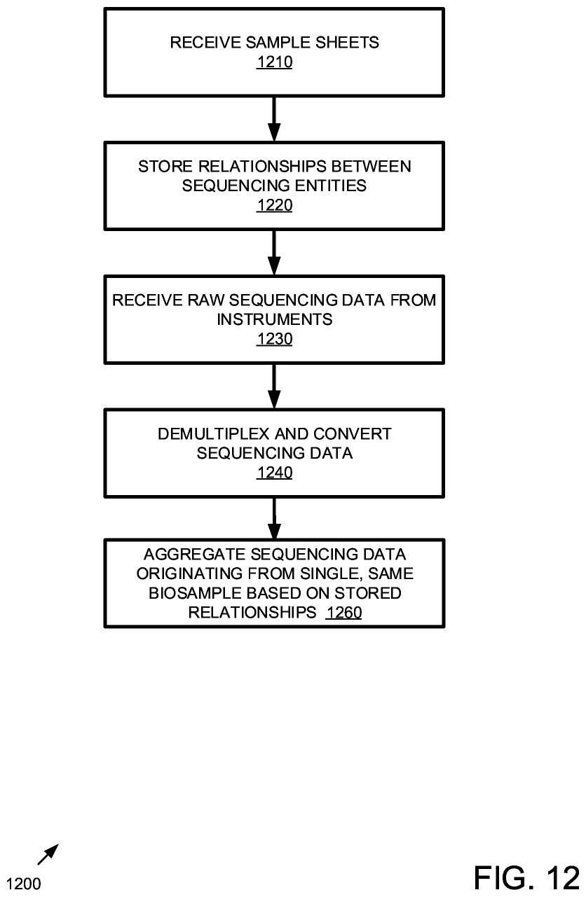

FIG. 12 is a flowchart of an example aggregation method 1200 showing details of how data relating to a particular biosample is identified as originating from a particular biosample, which can be used in any of the examples herein. Identifying which of the candidate biosample sequencing data sets originates from a particular biosample can comprise matching an index identifier associated with a particular biosample identifier with an index identifier indicated by a candidate biosample sequencing yield data set (e.g., detecting matches between the two). A match between index identifiers indicates that the data set originates from the particular biosample. In practice, other information (e.g., instrument identifier, lane identifier, and the like) can be used for correlation. As described herein, the index identifier can indicate an actual index sequence attached to the biosample during preparation and read by a sequencing instrument during sequencing. Therefore, when sequencing information is grouped by index identifier, it is possible to determine from which biosample the information originates if it is known which index was used for the biosample.

Additional information can be used for (e.g., to supplement) the matching process. For example, if a relationship is stored between a run identifier and the biosample identifier, identifying can comprise matching a run identifier of a candidate biosample sequencing yield data set with the run identifier stored in the relationship (e.g., along with the index identifier). A lane identifier can also be used for (e.g., to supplement) matching.

At 1210, a plurality of samples sheets for a particular biosample represented by a biosample identifier are received as described herein (e.g., by a sequencing orchestration environment).

At 1220, relationships between different sequencing entities are stored in computer-readable media based on the sample sheets. For example, relationships between the biosample identifier and a particular sample sheet can be stored. The sample sheet can contain other information such as a lane identifier and an index identifier, and such relationships between sequencing entities can also be stored.

At 1230, raw biosample sequencing data for a plurality of biosamples can be received from sequencing instruments into which information from the sample sheets were fed as input. Relationships between the sequencing entities can be supplemented. For example, upon finishing a run, the raw output data can then be associated with the instrument identifier, run identifier, and the like.

At 1240, the raw biosample sequencing data is demultiplexed and converted to a plurality of candidate biosample sequencing yield data sets. As described herein, such yield data sets are associated with respective index identifiers.

At 1260, the candidate biosample sequencing yield data sets originating from a single, same biosample is aggregated based on the stored entity relationships. For example, the candidate biosample sequencing yield data sets originating from the particular biosample can be identified as described herein, and such data sets can be aggregated into aggregated sequencing data yield for the particular biosample.

As described herein, an index identifier can be associated with the particular biosample in a sample sheet provided as part of a sequencing run for the particular biosample (e.g., and submitted to the sequencing instrument as part of the sequencing process). Or, a laboratory information management system (LIMS) can generate such a sample sheet for a sequencing run for the particular biosample. Or, the sample sheet can be generated based on information provided by a laboratory information management system.

As described herein, quality control and requeue functionality can also be incorporated, along with sequencing yield progress monitoring and automatic launching of an application when there is sufficient yield.

### Example 36—Example Sample Sheet

In any of the examples herein, a sample sheet can take electronic form and store a variety of information about a prepared biological sample, such as the biosample identifier, an index identifier indicating the index sequence associated with the prepared sample, on which lane the prepared sample is being sequenced within the instrument, and the like.

A biosample identifier can take variety of forms, such as a string identifier for the biosample, which is typically a bar code but can have any value.

The sample sheet can be edited directly, or an automated tool can be used to create, edit, validate, and manage sample sheets across one or more sequencing projects.

In practice, information from the sample sheet is converted into a suitable format for consumption by the instrument, and information from the sample sheet can be used to store relationships between sequencing entities as described herein. Also, when a sample sheet is passed to a particular instrument, an entity relationship can be created and stored between the sample sheet identifier and the instrument identifier of the particular instrument.

The actual information present in the sample sheet can vary by implementation. For example, a wide variety of information such as investigator name, project name, date, experiment name, workflow, manifest file, and the like can also be included. In some cases, more than one index identifier can be present.

A sample sheet can also specify a target amount of yield and an application to be automatically launched when the target amount of yield is acquired. As described herein, aggregation can compare against the specified target amount. As described herein, such target amount of yield and application to be launched can be stored in other locations, such as part of a biosample manifest or the like.

Although a sample sheet can be provided as part of the process of initiating a sequencing run, alternatively, the sample sheet can be generated based on information provided from a laboratory information management system (LIMS) that manages sequencing run information and other aspects of the sequencing workflow.

### Example 37—Example System Tracking Yield Progress

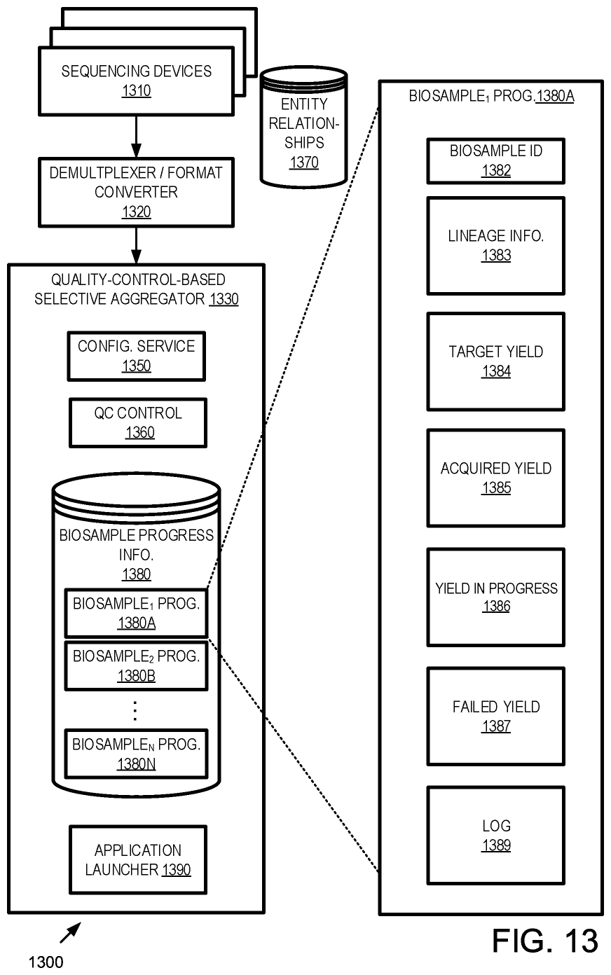

FIG. 13 is a block diagram of an example system 1300 tracking yield progress via a quality-control-based selective yield aggregator 1330 and can be implemented in any of the aggregation scenarios described herein.

In the example, a plurality of sequencing devices 1310 analyze a plurality of biosamples as described herein, outputting raw biosample sequencing data. Like the converter 140, a demultiplexer, data format converter 1320 accepts sequencing data of multiple libraries and outputs demultiplexed into a plurality of separate candidate biosample sequencing yield data sets (e.g., FASTQ files). Although a single demultiplexer 1320 is shown, in practice, a plurality of demultiplexers 1320 can execute in parallel on the same or separate computing systems.

The sequencing devices 1310 and the converter 1320 send digital events for consumption (e.g., by event subscribers) that indicate when processing has started (e.g., raw data has been received and is being demultiplexed and converted), and when the demultiplexing and conversion for a particular biosample sequencing yield data set is completed. The event can also include information that allows correlation of the incoming data with other information in the system to determine a match between a library, biosample, run, lane, and the like.

The demultiplexer 1320 and aggregator 1330 can execute on computing systems that are local to or remote from the sequencing devices 1310. For example, cloud computing scenarios can be supported.

As shown, the quality-control-based selective aggregator 1330 can include a configuration service 1350, quality control system 1360, biosample progress information 1380, and an application launcher 1390. Sequencing entity relationships 1370 stored in a computer-readable medium can be used to determine to which biosample (e.g., biosample identifier) yield from candidate data sets are to be applied and can represent various sequencing entities in an internal, digital representation.

The configuration service 1350 allows flexible configuration of the various features described herein. For example, different users may have different preferences that can be implemented by receiving such preferences and then implementing them.

The quality control system 1360 can perform the quality control processes described herein, such as implementing quality control thresholds to implement quality-control-based selective aggregation.

The biosample yield progress information 1380 includes biosample yield progress records 1380A-N for respective of the biosamples under analysis.

The application launcher 1390 can perform the automatic launching of an application as described herein (e.g., responsive to determine that there is sufficient yield).

An example biosample yield progress record 1380A is shown with details. In practice, the actual structure can differ (e.g., the log 1389 can be implemented separately from the record 1380A, elements can be combined, and the like).

In the example, a biosample identifier 1382 is used as a database key that allows tracking of a particular biosample across the sequencing device system. In practice, a friendly name and other information (e.g., description, tissue type, and the like) can be included.

The lineage information 1383 indicates details such as where the biosample came from (e.g., source organism, subject, or the like) as well as lineage within the system. Such information can refer to entities represented in the sequencing entity relationships 1370. For each biosample, the run and lane information for incoming yield can be tracked so that it can be traced back. Lineage for any sequencing entity can be tracked. For example, library and pool tracking can be implemented. Libraries and pools can also be used as keys in the database. Such an arrangement allows tracing upstream or downstream to know where the biosample yield came from (e.g., which run, which instrument, which lane, which library, which pool, and the like). Such an approach allows quality control per entity as described herein (e.g., a lane fails, and the yield associated with the lane is designated as failing quality control and not included in aggregation). Such quality control determinations are sometimes made after further analysis has been performed, so the lineage data can be maintained after aggregation and analysis are performed.

A target yield 1384 can also be stored for the biosample yield progress record 1380A. A target number of basepairs as described herein can be used to automatically trigger launching an application that performs further analysis on the sequencing data (e.g., for the particular biosample of the biosample id 1382). A pointer to or name of the application can also be stored. Alternatively, such information can be stored in a work order, and the progress record 1380 can refer to the work order.

The acquired yield 1385 indicates the actual current yield (e.g., yield amount in Gbp) for a particular biosample that has passed quality control. So, as incoming yield is detected, the acquired yield can be incremented to reflect. Failed yield that does not meet quality control can be excluded (e.g., filtered out).

The yield in progress 1386 indicates how much yield is in progress (e.g., yield amount in Gbp) for the particular biosample. As described herein, yield in progress can include both processing yield and pending yield.

If desired, failed yield 1387 can also be tracked to indicate how much yield has failed (e.g., yield amount in Gbp) for that yield that we ordered but never arrived, yield that did not meet quality control, or the like.

A log 1389 can also be maintained to indicate the various events that led to accumulation to yield, quality control failures, and a running log of activities engaged by the aggregator 1330 for the particular biosample of the biosample identifier 1382.

Integration between the aggregator 1330 and a library information management system (LIMS) can vary. A LIMS can be used to manage lab tasks, but some sequencing entities can be managed by the system incorporating the aggregator, such as flow cells, lane mapping, and data sets. Such parts of the sequencing workflow can be managed by a system incorporating the aggregator, and lineage information 1383 can come from various sources, including the LIMS if there is stronger integration with the LIMS.

### Example 38—Example Method of Tracking Yield Progress

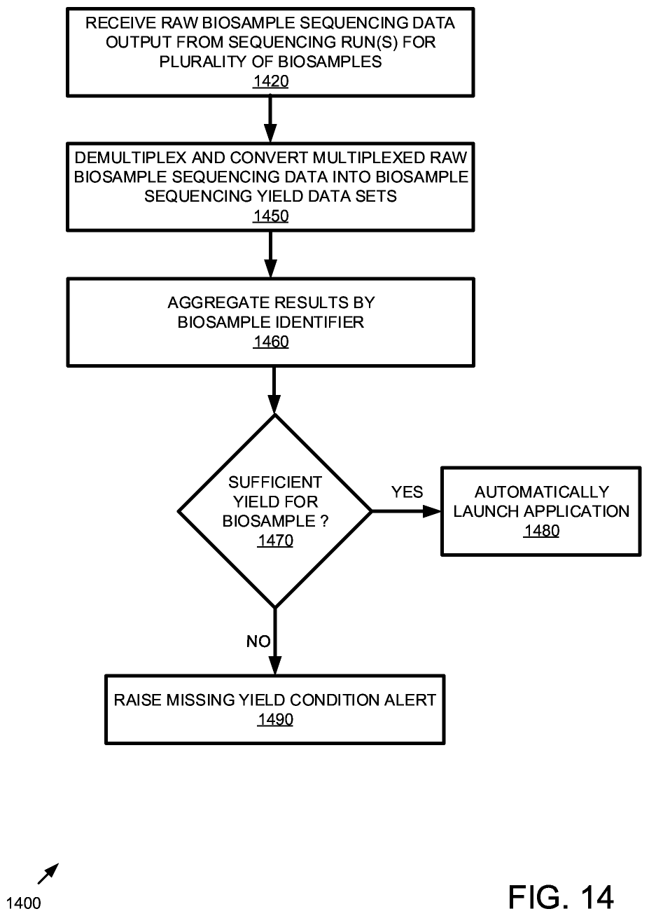

FIG. 14 is flowchart of an example method 1400 of tracking yield progress in a quality-control-based selective yield aggregation scenario and can be implemented, for example, in the systems of FIG. 1, 3, 5, 7, 9, 11, or 13. For example, a sequencing device system can comprise sequencing device system comprising a plurality of sequencing devices that output multiplexed raw biosample sequencing data for a plurality of input biosamples (e.g., comprising a particular biosample). As described herein, a target number of base pairs of sequence yield can be specified as sufficient for launching an application for further analysis of the particular biosample.

The system can also comprise one or more processors, and memory coupled to the processors, wherein the memory comprises computer-executable instructions causing the one or more processors to perform the process shown in FIG. 14.

The method 1400 can also be performed as a computer-implemented method or by one or more computer-executable instructions encoded on one or more computer-readable media that cause a computing system to perform the method. The method can also be performed in a sequencing environment comprising a plurality of sequencing instruments.

At 1420, raw biosample sequencing data output from sequencing runs for a plurality of biosamples are received (e.g., from a plurality of sequencing instruments or devices) as described herein. As described herein, such raw data can contain multiplexed data. The receipt of such data can be orchestrated by subscribing to events sent by the sequencing instrument or other infrastructure.

At 1450, the raw data is demultiplexed and converted into a plurality of candidate biosample sequencing yield data sets (e.g., FASTQ files). As described herein, such sequencing yield data sets are associated with single respective libraries and thus single respective biosamples associated with the libraries (e.g., including a run identifier, instrument identifier, or the like).

At 1460, sequencing results are aggregated by biosample identifier. In practice, a sequencing yield data set can be associated with a library identifier (e.g., barcode). Given the library identifier and sequencing run information associated with the dataset, it is possible to determine the biosample identifier for the yield data set. For example, the techniques described in conjunction with FIGS. 11 and 12 can be used. Yield data sets associated with the same biosample identifier are grouped together and associated with the biosample identifier. As described herein, aggregation can also take quality control into account so that selective aggregation is achieved (e.g., only those datasets meeting quality control are included in the aggregated data sets for the biosample).

Thus, aggregation 1460 can comprise identifying which of the candidate biosample sequencing yield sets originates from the particular biosample, and then aggregating the candidate biosample sequencing yield sets originating from the particular biosample into aggregated sequencing data yield for the particular biosample.

As described herein, a same identification technique can be used to identify and aggregate yield for both calculating an amount of yield (e.g., in Gbp) and group the actual yield results (e.g., sequences) together for further analysis.

At 1470, it is determined whether there is sufficient yield for a particular biosample identifier. Such a determination can determine whether the aggregated sequencing data yield for the particular biosample is sufficient, and the determination can comprise comparing a number of base pairs in the aggregated sequencing data yield for the particular biosample to the target number of base pairs.

For example, as incoming data sets originating from sequencing instruments are processed, they can be correlated and aggregated to biosample identifiers. The amount of aggregated yield for the biosample identifiers involved can be checked to determine whether yield is sufficient. For a particular biosample identified by a biosample identifier, the amount of aggregated yield (e.g., totaled, summed, or the like) can be compared to a target amount of sequencing yield to determine if it meets (e.g., is greater than, is greater than or equal to, or the like) a target amount of sequencing yield. Such a determination can be done as aggregation occurs, on a periodic basis, or on demand as described herein. In practice, running totals can be maintained to monitor progress as described herein.

Then, responsive to determining that there is sufficient yield, at 1480, a yield analysis application performing can be automatically launched and provided with the yield (e.g., sequencing yield datasets for the biosample identifier) as input. The application can then perform further analysis of the biosample with the aggregated sequencing data yield for the particular biosample.

Responsive to determining that there is not sufficient yield, a missing yield condition alert can be raised at 1490, indicating missing yield for the particular biosample. However, yield-in-progress can be accounted for to avoid over-requesting yield as described herein. Thus, determining that there is insufficient yield can comprise including yield-in-progress for the particular biosample. As sequencing activity continues, the process can resume with additional raw data received at 1420.

As described herein, a missing yield condition alert can also serve as a requeue alert in that the user may now request a requeue to acquire further yield and thus have sufficient yield for further analysis.

In practice, the tasks of 1420 and 1450 can be performed by separate components of the system. Therefore, the process can start with receiving biosample sequencing yield data sets and then aggregating such datasets at 1460.

### Example 39—Example Method of Determining Sufficient Yield, Accounting for Yield-in-Progress

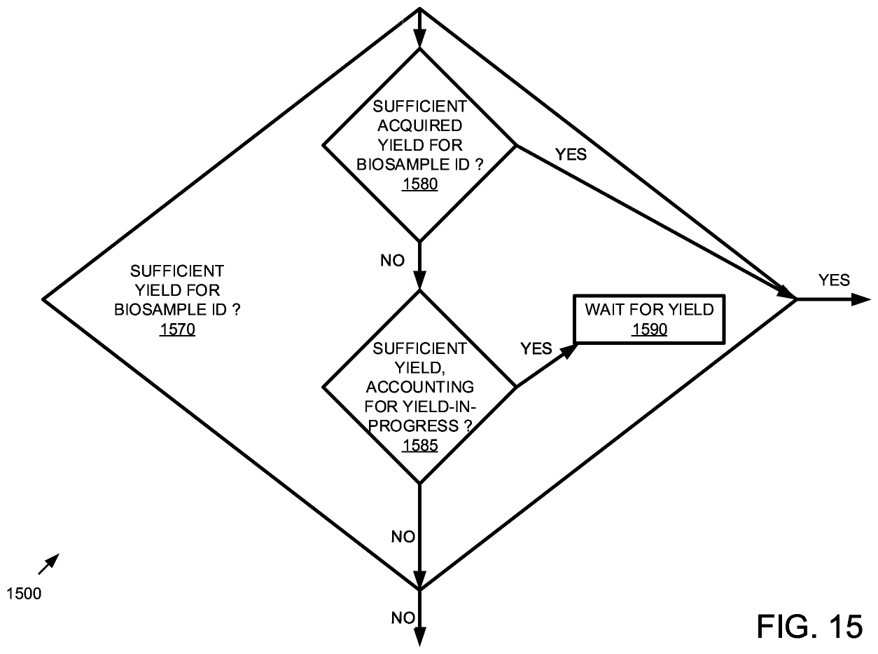

FIG. 15 is flowchart of an example method 1500 of determining whether there is sufficient sequencing yield for a biosample (e.g., identified by a biosample identifier), accounting for yield-in-progress, and can be used in any of the scenarios described herein relating to determining sufficient yield. For example, the method 1500 can be used to implement the decision at 1470 in FIG. 14. The method 1500 is one way of including yield-in-progress in calculations for determining whether enough yield has been requested for a particular biosample.

The overall determination 1570 of whether there is sufficient yield can include the method 1570. At 1580, it is determined whether there is sufficient acquired yield for the biosample identifier. As described herein, acquired yield can be the actual current yield (e.g., yield amount in Gbp) for a particular biosample that has passed quality control (e.g., the acquired yield 1385). A comparison can be made between acquired yield and target yield for a biosample (e.g., a comparison of a number of base pairs to the target number of base pairs). If the acquired yield is greater than or greater than or equal to the target yield, there is sufficient acquired yield.

Responsive to determining that there is sufficient acquired yield, the overall method can indicate a result of “yes” (e.g., there is sufficient yield).

Responsive to determining there is not sufficient acquired yield, at 1585, it is determined whether there is sufficient yield, accounting for yield-in-progress. For example, instead of including only acquired yield, yield-in-progress can be included in the comparison against the target yield. Yield-in-progress can include both pending yield and processing yield as described herein. Responsive to determining that there is not sufficient yield, even accounting for yield-in-progress, the overall method indicates a result of “no,” which can lead to a missing yield alert as described herein.

However, responsive to determining that there is sufficient yield when accounting for yield-in-progress, the determination can wait for additional yield. In this way, accounting for yield-in-progress can inhibit a result of “no” and the resulting missing yield alert. As described herein, such an approach can be particularly useful to avoid over-requesting yield.

As described herein, pending yield can eventually time out, at which point there may no longer be sufficient yield, even accounting for yield-in-progress.

### Example 40—Example Sufficient Yield

In any of the examples herein, sufficient yield (or “target” yield or “required” yield) can be stored as described herein to track yield progress. Such sufficient yield number can serve as a condition for further processing. For example, the sufficient yield can serve as a dependency or prerequisite for further processing. As described herein, the amount of yield considered to be sufficient can be set by a user requesting that the biosample be sequenced (e.g., via a work order as described herein).

### Example 41—Example Yield-in-Progress

In addition to acquired (or “actual”) yield that can take the form of generated sequencing yield data sets (e.g., FASTQ files), the system can account for yield-in-progress.

In any of the examples herein, yield-in-progress can include both pending yield (e.g., requested but not expired) and processing yield (e.g., undergoing demultiplexing and conversion) for a particular biosample.

Pending yield can be accounted for when a request is detected (e.g., by evaluating work orders or other data sources). In any of the examples herein, a timeout period can be set for pending yield so that it eventually times out, even if an explicit failure is not detected. Such a timeout period can be in minutes, hours, days, or the like. After the timeout expires, yield status can be updated to indicate that the request for yield has expired. Such yield can then be excluded from pending yield in yield-in-progress calculations.

Timeouts can be applied to both initial requests and requeues. The timeout can be set for a particular sequencing run responsive to determining that yield from any lane associated with the particular sequencing run has been received (e.g., when yield from any lane first shows up as having been sequenced).

In systems having greater integration between the laboratory information management system (LIMS), an explicit failure can be communicated to the system, which removes the yield as pending. For example, an indication can be received from the LIMS that a request for yield has completed, and responsive to receiving the indication, the tracked request can be marked as acknowledged (e.g., to prevent double counting it), whether for an initial request or a requeued request.

The actual amount of pending yield that is accounted for need not be exact. For example, a yield estimate can serve the purpose of avoiding excessive requests. For example, any request for yield can be assigned a default (e.g., user-configurable) yield amount, which then inhibits misleading indications that there is not sufficient yield. The yield-in-progress feature can utilize any placeholder that indicates yield acquisition is in progress, thus avoiding over-acquisition of yield.

Processing yield can include that yield that is expected to be uploaded to the system soon because it is undergoing demultiplexing and conversion (e.g., converted into FASTQ files).

Upon timeout of yield, a new determination of whether there is sufficient yield for the biosample can be made. If there is insufficient yield, a missing yield alert can be generated as described herein.

Yield-in-progress can be displayed (e.g., as “in-progress,” “pending,” “processing,” or the like) in a sequencing progress dashboard user interface so that progress is apparent to users.

By accounting for yield-in-progress in alerts and providing such information in user interfaces, the technologies can avoid over-request of yield. Without such a system, it can be commonplace to see that there is insufficient acquired yield and to request additional yield from the lab (e.g., via a work order). In fact, multiple such requests could result, leading to excessive over-request of yield. Thus, the technologies herein can conserve time and other lab resources that are otherwise wasted on acquiring unnecessary, excessive sequencing yield. Overlapping requests can thus be avoided.

### Example 42—Example Yield Aggregation Scenarios

In any of the examples herein, different terms can be used to identify different types of yield tracked by the system. A biosample preparation request can be a request to sequence a certain amount of data. Such yield is represented as “target yield” or “required yield.” The system can then track acquired yield, pending yield, and the like as shown. Expected yield can take the form of a sum of actual, processing, and pending yield.

### Example 43—Example Yield Aggregation Scenario Walk Through: QC Failure

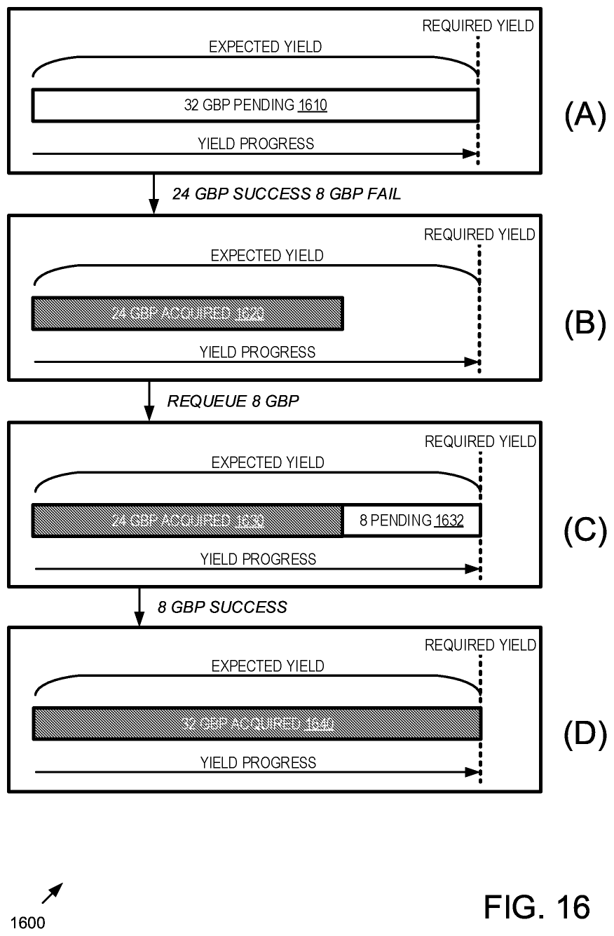

FIGS. 16A-D are bar graphs showing yield progress in an example quality-control-based selective yield aggregation scenario involving quality control failure. Such bar graphs can be displayed to represent yield progress for a particular biosample. In the example, a simple indication of “pending” is used for yield-in-progress. In practice, the actual numbers can vary greatly, and the initial requested yield can exceed the target yield.

At 16A, 32 Gbp have been requested for a particular biosample and are represented by the bar graph 1610.

During sequencing, 24 Gbp were successfully sequenced, but 8 Gbp failed quality control metrics. So, in FIG. 16B, the acquired yield 1620 is shown, but some yield is missing (e.g., there is not sufficient yield to meet the required, target yield).

Having detected the missing yield and raised a missing yield alert, the system receives a requeue request. At 16C, the pending yield 1632 is shown alongside the acquired yield 1630

Eventually, the 8 Gbp are successfully sequenced and meet quality control. There is now 32 Gbp or acquired yield 1640, which meets the target yield. Accordingly, a yield analysis application can be automatically launched and provided with the yield as input.

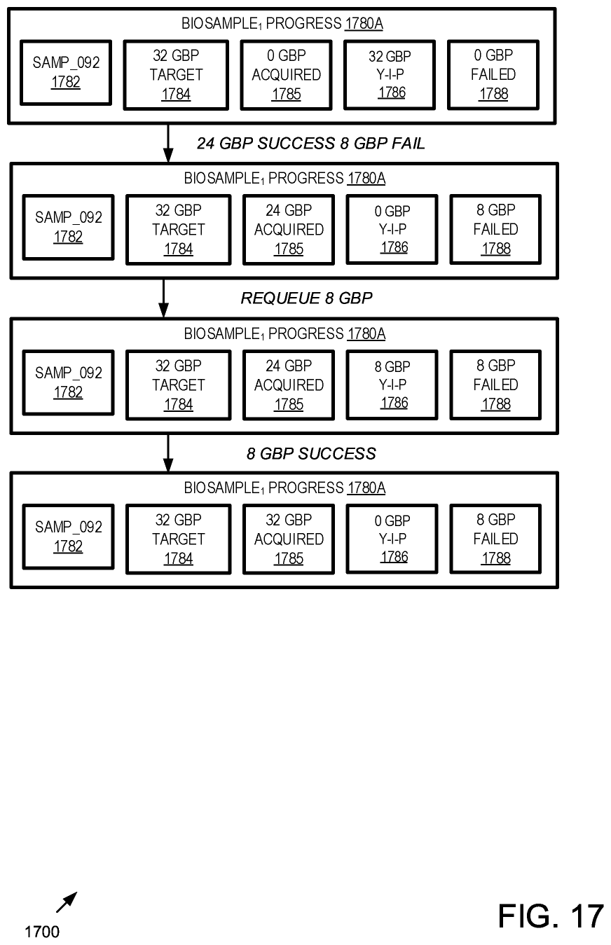

FIG. 17 shows an internal, electronic representation of yield progress in the scenario of FIGS. 16A-D. There are four quantities tracked internally by the system in a biosample progress data structure 1780A. Paralleling the scenario of FIG. 16, first the data structure 1780A stores an indication of the biosample identifier 1782, the target yield 1784, the acquired yield 1785, the yield-in-progress 1786, and the failed yield 1788. After some yield fails quality control, an alert is triggered, leading to a requeue and eventual acquisition of the target yield.

The yield-in-progress 1786 can serve as a placeholder for yield that is requested but not yet acquired.

The data structure 1780A can be used to track progress, generate a dashboard, and automatically launch an application upon successful acquisition of sufficient yield. Although an actual number of expected yield in Gbp is shown in the example, such a placeholder could take different forms, such as a simple indication that a run is in progress, the number of runs in progress, a default yield per run (e.g., configurable per user), or the like.

### Example 44—Example Yield Aggregation Scenario Walk Through: Expired Yield

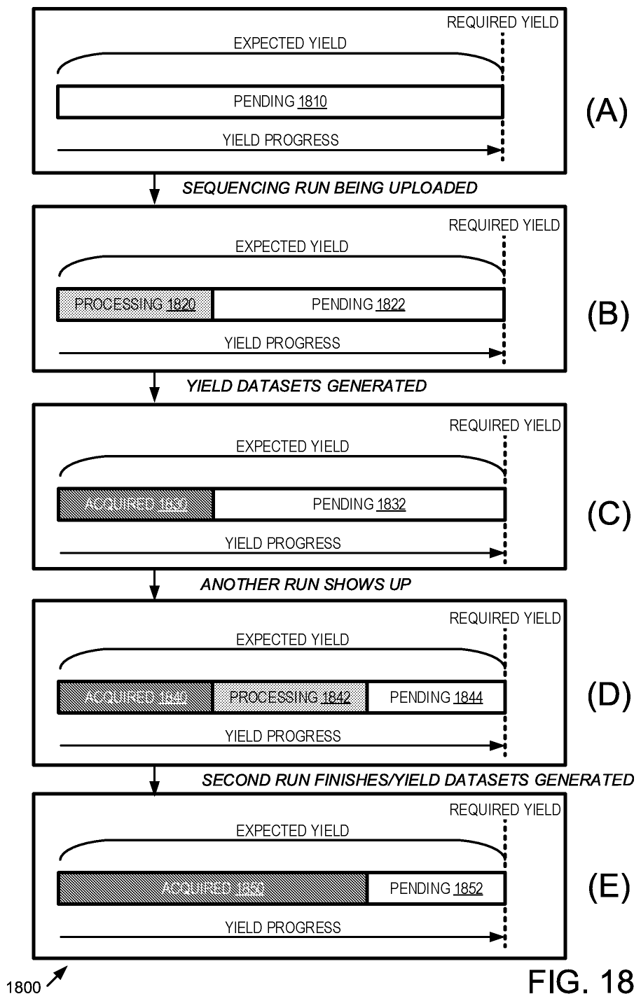

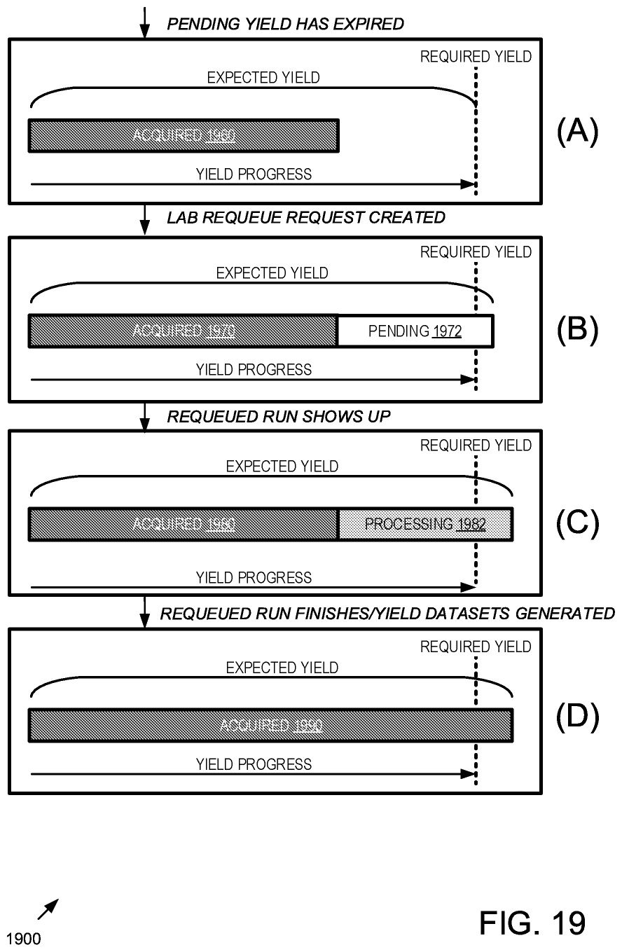

FIGS. 18A-E and 19A-D are bar graphs showing yield progress in an example expired yield scenario. Such bar graphs can be displayed to represent yield progress for a particular biosample. In the example, yield-in-progress is represented as either “pending” or “processing.” In practice, the actual numbers can vary greatly; as shown, the requested yield can exceed the target yield when yield-in-progress is included.

At 18A, there is an amount of pending yield. A work order is put in through a biosample workflow .csv file. Yield is all pending at this point because the order has just been initiated.

At 18B, a sequencing run is being uploaded to the system. Yield is being processed as .bcl files are converted to FASTQ files.

At 18C, FASTQ data sets are generated. Yield is now counted as actual yield that can be used for application input when using a biosample.

At 18D, another run has shown up. The totals show that it is expected that there will be sufficient yield to meet what is required.

At 18E, the second run finished (e.g., is converted to FASTQ format).

At 19A, pending yield has expired. After a configurable time period as described herein, the original request expires and pending yield is set to zero. The system now triggers a missing yield status on the biosample to notify the user to ask for more.

At 19B, a user creates a lab requeue request for more yield. The system now shows the yield as pending because it is expected that the lab will fulfill the extra work order.

At 19C, the lab put the requeued sample onto another run, which is being uploaded to the system. In the example, the expected yield exceeds the minimum required amount.

At 19D, the original work order and extra work order are now complete. The yield analysis application can launch automatically if it only depended on having enough sequencing data to be present.

The different kinds of yield can be represented in digital form internally similar to that as shown in FIG. 17.

### Example 45—Example Expected Yield Matching System

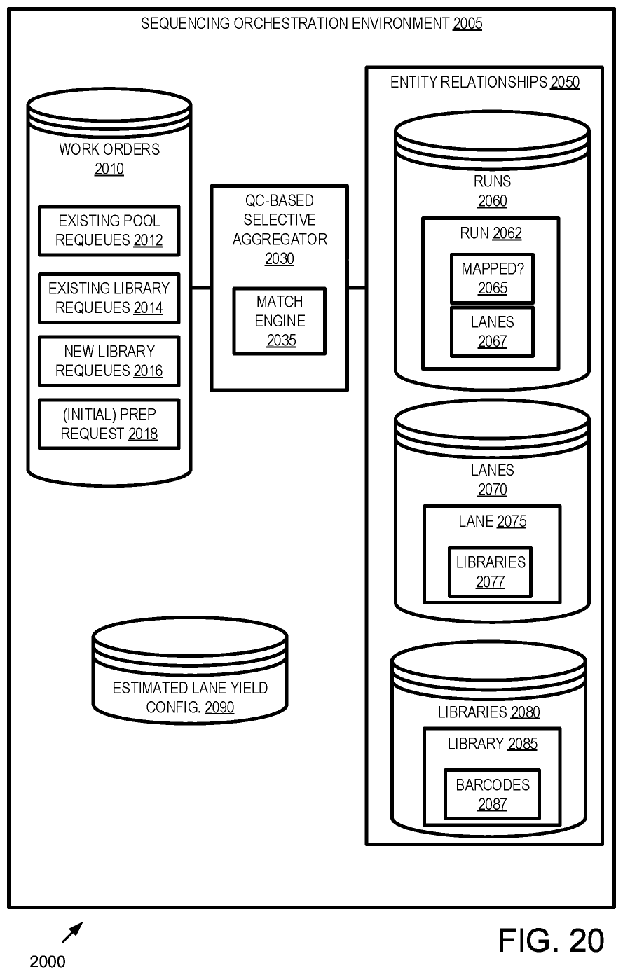

FIG. 20 is a block diagram of an example system 2000 matching expected yield from sequencing runs to lab requests for tracking yield progress and can be implemented in any of the systems described herein that track yield-in-progress. In practice, matching can be used as part of monitoring yield progress in that matching can enable determining how much yield is in progress, allowing accurate estimation of yield-in-progress, including pending or processing yield.

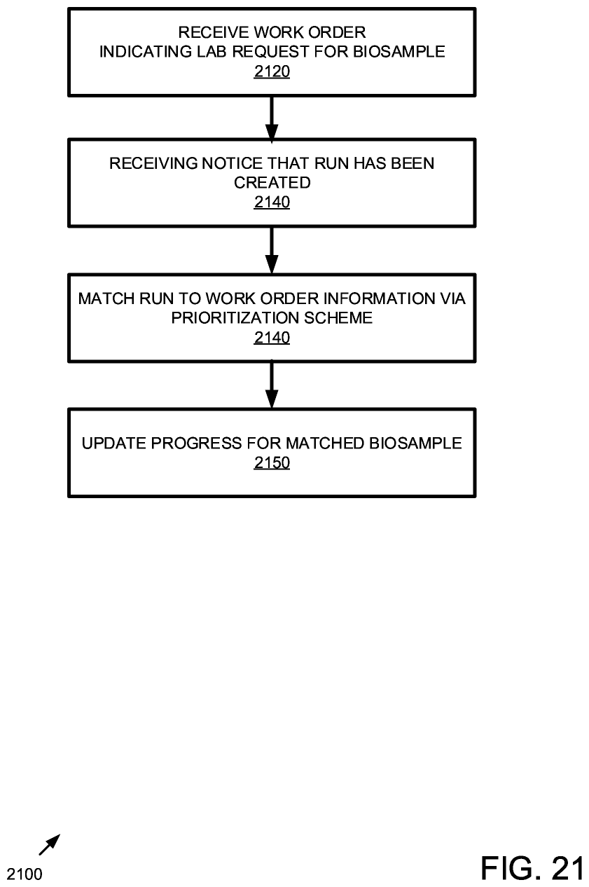

In the example, a quality-control-based selective aggregator 2030 can execute in any of the environments described herein, such as a sequencing orchestration environment 2005. A match engine 2035 within the aggregator 2030 can match work orders with lab requests, including existing pool requeues 2012, existing library requeues 2014, new library requeues 2016 and prep requests 2018. Such an engine 2035 can perform the method of FIG. 21 or the matching acts therein.

When the environment 2005 detects that a new sequencing run has begun, a message can be sent that can be detected by the aggregator 2030. Thus, a run can show up before it is completed. Because it can take a significant amount of time for the run to complete, it is useful to account for the yield expected from the run as part of yield progress as described herein.

Various entity relationships 2050 can be stored in computer-readable media, including information on runs 2060, lanes, 2070, libraries 2080, and others.

In addition, a per-user configurable estimated lane yield configuration 2090 can be set by users to indicate the amount of expected yield (e.g., Gbp for a lane), which can be incorporated into yield progress when a lab request is matched to a run. If such information is not present, statistics can be consulted to estimate expected yield. Or, a simple default value (e.g., a constant indicating a number of Gbp, such as MaxProjectedYieldlnGbp) can be used to avoid too many missing yield alerts.

An entry 2062 for a particular run can comprise an indication 2065 of whether or not the run has been mapped yet, and with which lanes 2067 it is related.

An entry 2075 for a particular lane can comprise an indication 2077 of the libraries associated with the lane.

An entry 2085 for a particular library can comprise an indication 2087 of the barcodes (e.g., index sequences) associated with the library.

Other tables can include additional information. For example, library-biosample associations can be maintained.

### Example 46—Example Expected Yield Matching Method

FIG. 21 is a flowchart of an example method 2100 of matching expected yield from sequencing runs to lab requests for tracking yield progress and can be implemented, for example by the system of FIG. 20 (e.g., the match engine 2035) or other systems that track yield progress.

At 2120, a work order is received indicating a lab request for a particular biosample. In some cases, the work order is related to a requeue. A relationship between the requeue and the work order can be stored by the system. For example, as part of the requeue alert user interface, an indication can be stored indicating that resulting work orders are related to the requeue. As described herein, lab requests can be existing pool requeues, existing library requeues, new library request, and initial prep requests.

Subsequently, a notice can be receive that a run has started. Such a notice can take the form of a message from the system. An entry in stored sequencing entities can be created to represent the sequencing run. As described herein, such entity relationships can include relationships between a library, sequencing instrument, run, lane, and the like.

At 2140, the run is matched to work order information via a prioritization scheme, and the biosample involved (e.g., biosample identifier) is thus determined. In practice, a lane-by-lane match can be performed (e.g., a particular lane for a particular run is matched to a particular work order). A prioritization scheme can check requeues before checking initial sequencing runs as described herein. The lineage information used for aggregation can be used for matching purposes. For example, as described herein, index sequencing information can be utilized for matching along with other information.

At 2150, after finding a match, the progress for the particular biosample is updated as described herein. For example, acquired yield, yield-in-progress, failed yield, and the like can be updated. In practice, an estimated amount of yield can be calculated based on user preferences, statistics, or the like.

The method 2100 can be used for requeues or initial requests. In any of the examples herein, a requeued request for yield can be tracked, which can comprise matching the requeued request to an active sequencing run, and predicted yield from the active run can be included in yield-in-progress for the particular biosample of the requeue. Matching can prioritize requeues over initial requests.

### Example 47—Example Implementation of Estimating Expected Yield

In any of the examples herein, expected yield can be estimated using a variety of techniques as part of an overall design to account for yield progress. Predicted incoming yield from sequencing runs (e.g., whether finished or not) can be matched to outstanding lab requests for biosamples. By accounting for the requested yield using estimated incoming yield, the system can more accurately determine the amount of yield expected to be seen in the future (e.g., pending yield) and thus determine when a biosample is missing yield.

The following lab requests can be associated with incoming yield from sequencing runs:

Existing Pool Requeues:

Existing pool requeues ask for more yield of an entire library pool. They are typically mapped to one or more lanes containing the entire pool. The pool associated with the lane in the sequencing run must match the pool in the requeue exactly. If a lane with a pool is found, and there is an outstanding lab requeue for the pool, it is very likely that the lane is associated with the requeue. The entire lane can be designated as associated with the lab requeue, and it can be prevented from matching any other type of request.

Existing Library Requeue:

Existing library requeues ask for more yield of a specific library associated with a biosample, but do not specify the library pool that must contain the library. Therefore, the library could come in an existing pool, a new pool that has not been encountered by the system, or even as the entire content of a lane. To make a match, the incoming lane must contain an exact match for the library that was requested. Such a type of match is partial, in that other contents of the lane can also match other requests for different biosamples simultaneously.

New Library Requeue (a/k/a Biosample Requeue):

This type of requeue asks for more yield for a biosample using a specific library type (e.g., prep kit). It does not specify the library to be used to provide the additional yield. Therefore, the matching library could be an existing library or a new library, as long as the library type (e.g., prep kit) matches the request. It could come in an existing pool or a new pool.

Prep Requests:

Prep requests represent the initial request to the lab to produce yield for the biosample. They are similar to the New Library requests in that they only specify a library type (e.g., prep kit). The matching library could come in any form as long as the type matches the requested type.

Asynchronous Message

The system can use an asynchronous message (e.g., SatisfyRequestMappingsWithLanes) associated with a run to trigger the lane-to-lab-request matching process when a new run is detected. Before the matching is performed, the run will have lane-library mapping established (because the matching needs to know what biosamples to match against). In order to match the correct amount of yield against the lab request, the system can also determine how much yield each lane in the sequencing run will provide. This can happen by

1. Expected Yield Per Lane Configuration: A configuration setting that users can provide that specifies the amount of yield each lane of a matching run will provide for yield matching purposes; or

2. Using Sequencing Statistics: If an expected yield per lane configuration for the run is not found, the matching can rely on the MaxProjectedYieldInGbp value associated with the lane. The value is computed based on the Interops of the run as part of the GenerateSequencingStats asynchronous message.

The logic can be as follows:

1. When lane-library mapping are established for an arriving sequencing run (e.g., either through LIMS or via instrument sample sheet), the system registers a SatisfyRequestMappingWithLanes asynchronous message to check if it can process the run and complete the mapping process.

2. When the run's interops are first parsed and the MaxProjectedYieldInGbp for each lane is calculated, the system also registers a SatisfyRequestMappingWithLanes asynchronous message in case the run needs sequencing states in order to perform the mapping.

The messages can come at any time in any order to the message consumer, therefore, the message consumer only processes the message after the run has established lane-library mappings. The run entity has a property that is set to so indicate. Also, the message consumer checks to see if there is no matching expected yield per lane configuration for the run. If so, sequencing statistics are generated for the run. This is determined by the run having non-null sequencing statistics. If there is a matching expected yield per lane configuration, it is not necessary to wait for sequencing statistics, and the association can proceed immediately.

Because it is possible that there could be multiple SatisfyRequestMappingsWithLanes asynchronous messages triggered for the same run from different places, the following can ensure that the mapping is performed only once for a given run:

1. The system can detect if multiple consumers are processing a message for the same run simultaneously. If detected, the message can be placed back in the queue with a delay for later processing

2. A property on the run can be used to detect when the run has successfully performed SatisfyRequestMappings processing so that it is not processed again.

A goal can be to satisfy pending lab requests with incoming yield from sequencing runs as soon as possible (e.g., in case the run fails before sequencing stats are computed). So, the SatisfyRequestMappingsWithLanes message processing can be performed immediately when lane-library mappings are first established. If there is no expected yield configuration, the system can wait for sequencing stats to be generated before proceeding. Such an approach can ensure that pending yield is adequately accounted for even if the run fails in the early cycles before interops are parsed, if the expected yield per lane value is known.

Because there may be multiple SatisfyRequestMappingsWithLanes messages for the same run, duplicate processing of the message for the same run can be prevented.

Keeping Track of Associations Between Lab Requests and Incoming Sequencing Runs

An entity called “LaneSatisfiesRequestMapping” can be used to keep track of the lab requests that have been associated with a given lane. The entity associates the lane with either a LabRequeue or a PrepRequest.

For existing pool requeues, there need only be a single LaneSatisfiesRequestMapping entity per lane because the entire lane can be associated with a single pool requeue.

For other types of lab requests, there can be multiple LaneSatisfiesRequestMapping entities per lane because a single lane can be matched to multiple lab requests simultaneously (e.g., one lane can match a single lab request for a given biosample, but it can match multiple lab requests for different biosamples).

Such LaneSatisfiesRequestMapping entities can be used to compute the amount of yield that each lane contributes to both LabRequeues and PrepRequests during the biosample yield calculation for each sample.

Enhancing Prep Request Timeout Period

The current Prep Request timeout can start with the first lane associated with the Prep Request is found. However, such an association may not be created until after a run has sequencing statistics. If the run fails before the sequencing statistics are generated, the association may not be created, and the Prep Request may never expire.

For Prep Requests specifically, a separate backup approach can be used for time out: If any run associated with the biosample/library type is detected, the oldest such run's creation data can be used as the start of the timeout period for the Prep Request because it is typically associated with the prep request. Such an approach can correct problems with the need to use sequencing statistics when satisfying prep requests or lab requeues.

Such a problem can be corrected with an implementation that matches yield from a run against the prep request before sequencing stats are generated. The logic can be preserved for cases when the expected yield per lane is not configured, and the sequencing stats are relied upon.

Expiring Lab Requests after a Timeout Period

To expire lab requeues after a timeout period, an approach similar to that used for the expiration of Prep Requests can be used. In general, lab requeues are higher priority than initial prep requests, so they are expected to be processed by labs in a reasonable time period.

The date and time when a lab requeue is marked as acknowledged can be recorded. The configurable timeout period can start when the requeue is acknowledged (e.g., AcknolwedgedOn date).

If a lab requeue expires, it no longer contributes to the pending yield of the biosamples associated with the requeue, but it can still receive incoming yield from sequencing runs and eventually move to fulfilled status.

In some implementations, only acknowledged requeues can expire. A user can manage the pending lab requeue to indicate that it is canceled or expired.

Expected Yield Per Lane Configuration Entity

A database table can be used to store a per-user configuration of expected yield per lane values:

ExpectedYieldPerLaneConfiguration (table/entity):

| Column | Data | | |
|---|---|---|---|
| Name | Type | Nullable | Description |
| Id | bigint | No | Primary Key |
| Owner | bigint | No | Foreign Key to user associated with the |
| UserId | | | entry |
| Platform | string | Yes | Maps to the PlatformName enum |
| Name | | | matching this configuration entry |
| | | | or null if not matched |
| Instrument | String | Yes | Maps to InstrumentType enum matching |
| Type | | | this configuration entry or null if not |
| | | | matched |
| Instrument | Bigint | Yes | FK Maps to specific instrument matching |
| Id | | | this configuration entry or null if not |
| | | | matched |
| Barcode | String | Yes | Optional barcode mask associated with |
| Mask | | | this configuration entry (regular |
| | | | expression) |
| Expected | Bigint | No | The expected yield per lane for runs |
| Yield | | | matching this configuration |
| PerLaneBp | | | |

To avoid unnecessary additions to the Lane entity, the existing MaxProjectedYieldlnGbp field of the Lane can be used. Such a field represents the maximum projected yield for each lane found during the entire run. For the feature, the value can be initialized to the “expected yield per lane” value based on configuration when it is available. Because it is a maximum, it sets the floor for the value for each run.

API Changes

An API can be provided to allow users to create, view, update, and delete Expected YieldPerLaneConfiguration entries:

POST/v2/expectedyieldperlaneconfigurations—Create

PUT/v2/expectedyieldperlaneconfigurations/{id}—Update

GET/v2/expectedyieldperlaneconfigurations—Get list

DELETE/v2/expectedyieldperlaneconfigurations{id}

Processing

When a run is first created, the system looks for a matching ExpectedYieldPerLaneConfiguration for the fun in the following order:
InstrumentID (80 points)
BarcodeMask—regular expression match (40 points)
InstrumentType (20 points)
InstrumentPlatform (10 points)

If an entry does not match any of these items, it has 0 points and is not used. This means that empty configuration entries do not match any runs.

If more than one matching configuration entry for the run is found, the entry with the most matching points is used for the particular run.

If a match is found, the lane is updated with the matching ExpectedYieldPerLaneBp value by setting the MaxProjectedYieldInGbp value to match the ExpectedYieldPerLaneBp value. Unit transformation can be performed. Otherwise, the MaxProjectedYieldlnGbp value is left at its current value (e.g., presumably set when the Interops were parsed and the sequencing statistics were generated).

When computing biosample yield, by default, the system can use the MaxProjectedYieldInGbp value for calculating ProcessingYield instead of ProjectedYield, unless a user configuration setting is set to use ProjectedYield for ProcessingYield. Such an approach gives runs stability while the run is sequencing and can avoid premature Missing Yield determinations for a biosample. Such an approach can be useful for avoiding many Missing Yield events while a run is sequencing.

Matching Expected Yield from Run to Lab Requests

The following logic can be used to match expected yield from the run to existing lab requests. The system can be configured so that only LabRequeues that are Acknowledged and not yet Fulfilled can receive incoming yield from a given Lane. A LabRequeue can be come Fulfilled from one lane in a sequencing Run and should not be considered for other lanes in the same sequencing run after this happens. For consistency, lanes can be matched against requests in increasing order of Lane number.

Prep requests can receive incoming yield from sequencing runs regardless of whether the requested yield from the prep request has already been matched or not.

The order of consideration can be based on whether a matching ExpectedYieldPerLaneConfiguration entry was found or not:

1. If a configuration entry was found, the lab requests are ordered “oldest first” for consideration. Ordering only applies within a given priority level. “Oldest first” is used because expected yield configuration is accurate and should fully account for requested yield with sequencing yield, so it makes sense to try to fulfill older requests within a given priority level first before considering newer requests.

2. If a configuration entry was not found, the lab requests are ordered “newest first” for consideration. Ordering only applies within a given priority level. “Newest first” is used because the expected yield from the sequencing statistics is typically not very accurate and likely to underestimate the amount of yield. So, it makes sense to try to match newer requests with new sequencing data assuming that older, unfulfilled requests may have only been partially matched, and that is the reason they are not yet fulfilled.

For each lane in the run (e.g., in the order of increasing lane number 1-n):

Priority Level 1—

Existing Pool Requeues take precedence over other lab requests and are matched first until fully fulfilled (e.g., regardless of the dates of the other requests). Matching can require that the lane have the exact pool associated with the existing pool requeue in order to be a match.

Only a single Existing Pool Requeue can be matched to a given lane. If an Existing Pool Requeue is matched to a lane, no other lab request can be matched to the lane.

Because the entire lane matches a single existing pool lab requeue, the entire yield from the lane can be associated to the Existing Pool Requeue, regardless of how many libraries the pool contains.

The requeue may match multiple lanes until it is fulfilled.

Priority Level 2—

Existing Library and New Library Requeues are considered next and take precedence over prep requests for matching purposes. Matching can require that the lane contain the exact library in order to match Existing Library Requeues, and that it contain a Library for the biosample of the same LibraryPrep in order to match New Library Requeues for a given biosample PrepRequest.

Only a single lab requeue for a given biosample can be matched to a given Lane. Requests for different biosamples can match the same lane simultaneously.

Lab requests in this priority level are considered in their date order until they are fulfilled. They can match multiple lanes from the same run.

For lanes with pools, it can be assumed that the yield from the lane is evenly distributed among the libraries in the pool for yield assignment purposes. Such an approach means that each matched lab request will only get a portion of the lane yield associated with it. For example, if the pool has three libraries, each match request gets one third of the yield for the entire lane.

Priority Level 3—

Prep Requests for biosamples are considered last. For matching, it can be required that the lane contains a library for the biosample of the same LibraryPrep as the Prep Request.

If a lab requeue for a given biosample was previously matched to a given Lane, a prep request from the same biosample should not be matched to the lane. Requests for different biosamples can match the same lane simultaneously.

Prep requests are consistently associated with a matching lane at this level, even if the prep request required yield is already fully matched. In this way, lanes containing the biosample can be matched with something.

For lanes with pools, it can be assumed that the yield from the lane is evenly distributed among the libraries in the pool for yield assignment purposes.

Although the logic may assume that a pool will only contain a single library associated with a given biosample, it can be updated for other scenarios.

### Example 48—Example Incoming Yield Matching Internal Representation

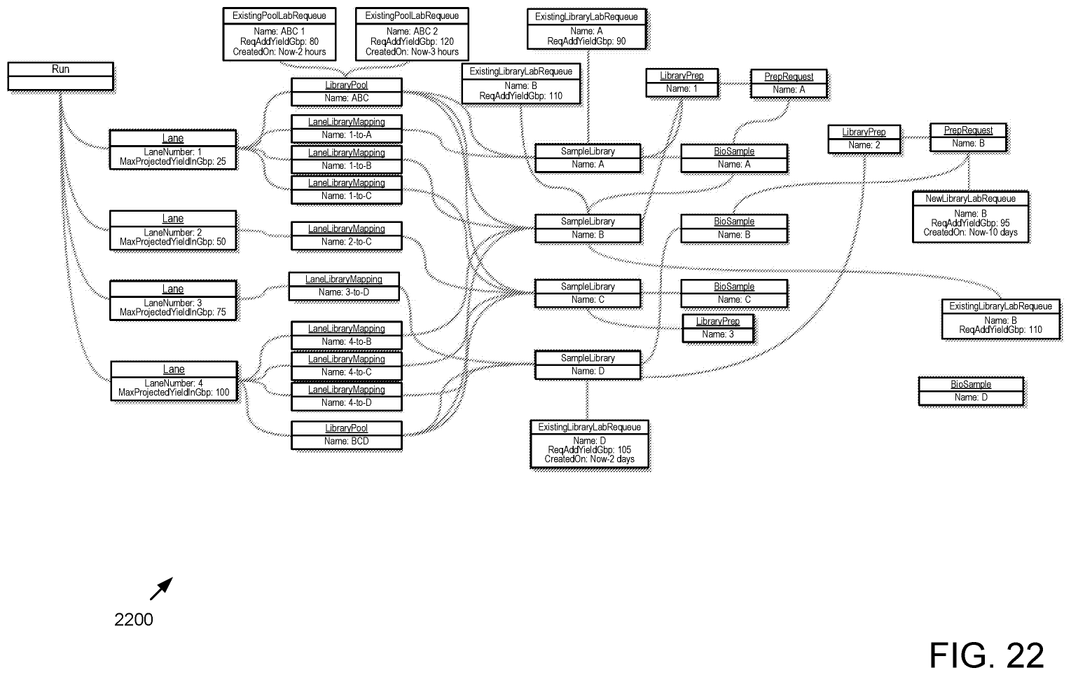

FIG. 22 is a block diagram of an example internal electronic representation 2200 of relationships between sequencing entities for use during yield matching. As shown, relationships between a particular run, one or more lanes, libraries, and samples can be maintained.

Requeues can be represented along with when the requeue was created, leading to more accurate matching of incoming yield to requeues, which then results in launching the associated yield analysis application sooner.

### Example 49—Example Distribution of Tasks

In any of the examples herein, various tasks can be performed by different components or hardware of the system. For example, for those implementations involving receiving raw data and demultiplexing/converting such data, such work can be performed by a different component than the component aggregating results. For example, a sequencing instrument can include hardware for performing additional tasks beyond simply outputting raw sequencing data.

### Example 50—Example Implementation

The technologies described anywhere herein can be implemented into any of a variety of sequencing orchestration environments for interacting with the data. For example, the technologies can be integrated into the ILLUMINA BASESPACE Sequence Hub system provided by Illumina, Inc.

Although simple linear scenarios are described in some examples herein, a sequencing orchestration environment can support ongoing maintenance of sequencing results. For example, a user can arbitrarily pick and choose to add further sequencing data that is not relevant to a particular automated task. Data used for one yield analysis application can be re-used and/or supplemented and analyzed by the same or another yield analysis application.

### Example 51—Example Comprehensive Implementation

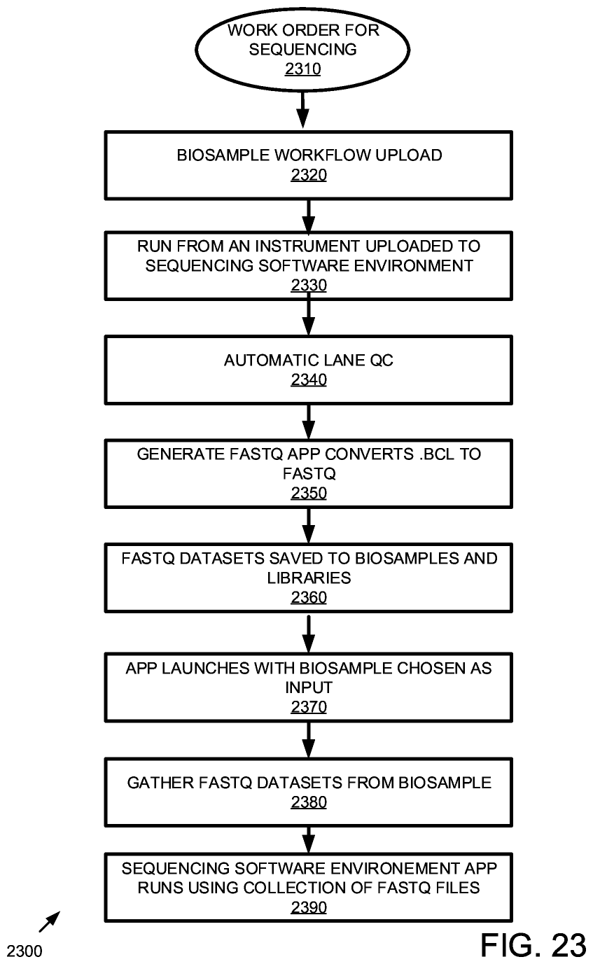

FIG. 23 is a flowchart of a method 2300 of an example implementation of the technologies into a comprehensive sequencing orchestration environment and can be used to achieve any of the aggregation technologies (e.g., a yield aggregator) described herein.

At 2310, a work order for sequencing is received, initiating the sequencing work flow. A user decides that they wish to sequence a biosample, and a certain amount of data is needed to run a successful analysis. The data (e.g., yield) may come from multiple libraries, pools, or instruments.

At 2320, the biosample workflow is uploaded to the environment. The workflow includes a work order for the biosample to attain a certain amount of sequencing yield and launch a specific yield analysis application when reaching the yield.

At 2330, a connected sequencing instrument uploads .bcl files to the environment.

At 2340, statistics from the sequencing run are evaluated on a lane-by-lane basis, where automatic thresholds determine a pass or fail status. Failures will excluded data in downstream aggregation.

At 2350, a run's .bcl files are converted to FASTQ files by an environment application automatically. The files are saved as FASTQ datasets, which are the source of yield for biosamples.

At 2360, the newly created FASTQ data sets are linked to biosamples and libraries.

At 2370, a user may choose one or more biosamples as an input to a sequencing orchestration environment application.

At 2380, the environment finds all non-failed FASTQ datasets linked to the chosen input biosample(s). Other linked entities to the biosamples can be checked for failure status, which may exclude more data sets.

At 2390, the yield analysis application uses the FASTQ files gather together as input to its algorithm(s) to produce outputs. The outputs may be used for further downstream analysis.

### Example 52—Example Work Order Implementation

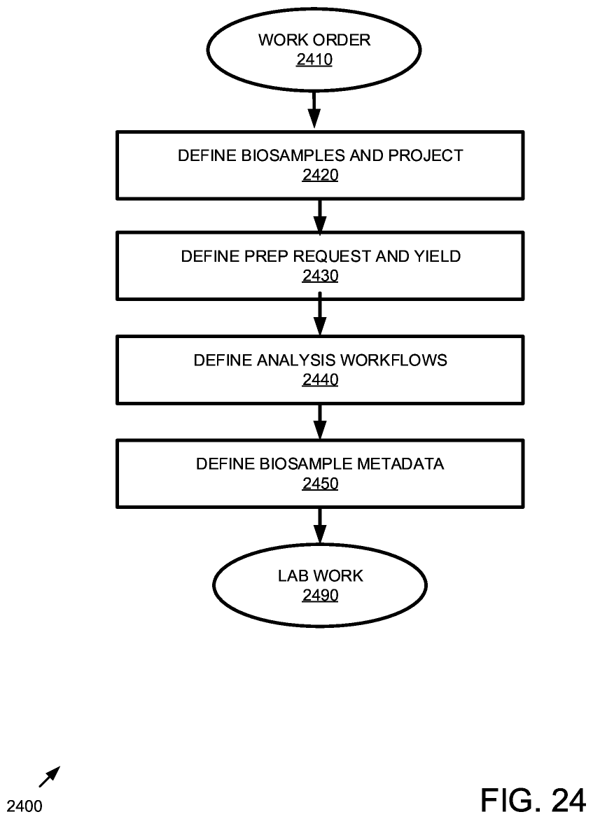

FIG. 24 is a flowchart of an example method 2400 of implementing work orders and can be implemented in any of the examples herein involving work orders, including 2320 of FIG. 23.

At 2410 a biosample workflow .csv template is downloaded. A user can fill out the form to define the work order and what is to be automated.

At 2420, for the work order, the biosamples can be named, and the default project can be specified. Applications processing the resulting sequencing data can write data to the default project.

At 2430, a prep request is added. The prep request can indicate the library prep kit to use for biosample preparation. It can also define the target yield needed to run the application. It can be the original work order request for the lab to produce a certain amount of sequencing data.

At 2440, analysis workflows can be defined. Such workflows can be application templates for automation. They can be scheduled ahead of time with the .csv upload and launch when the dependencies (e.g., acquisition of yield) are met.

At 2450, meta data key-value pairs can be included if desired to add more information to biosamples. Such data need not affect yield or application launches.

After the .csv file is uploaded to the sequencing orchestration environment, the specified biosamples, projects, and analyses are created. The lab can begin work on meeting the yield.

### Example 53—Example Lane-Based Quality Control

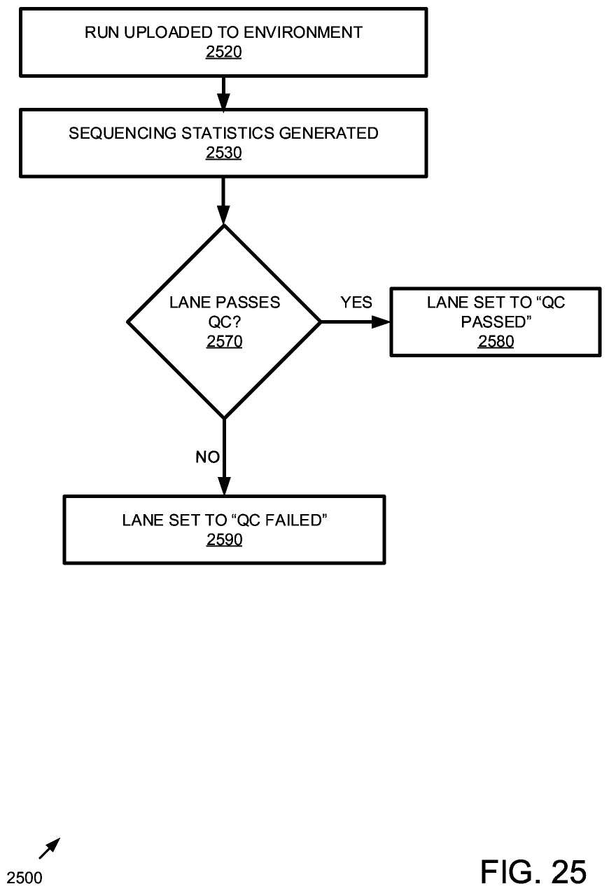

FIG. 25 is a flowchart of an example method 2500 of implementing quality control in a sequencing data aggregation scenario by sequencing lane and can be implemented in any of the examples herein involving quality control, including 2340 of FIG. 23. Although lane-based quality control is shown, other sequencing entities can be used in addition to or instead of lanes.

At 2520, the sequencing instruments uploads .bcl files and other run files to a user's account in the sequencing orchestration environment.

At 2530, using the interop files from the run upload, the environment determines statistics about the quality and yield of each flowcell lane.

At 2570, based on a user's setting that store their thresholds on specific metrics to determine the quality of each lane, the environment can set a lane to “QC Passed” at 2580 if thresholded metrics are passed. Failure results in setting to “QC Failed” at 2590. A user can view the automatically set lane status and manually override it. Setting a lane to “QC Failed” excludes data produced in that lane for biosamples of the lane.

The environment can use the .bcl files from the run to generate FASTQ files. The application that generates FAST files can be unaffected by the lane status, which affects data aggregation at a later step.

### Example 54—Example Quality Control Across Sequencing Entities

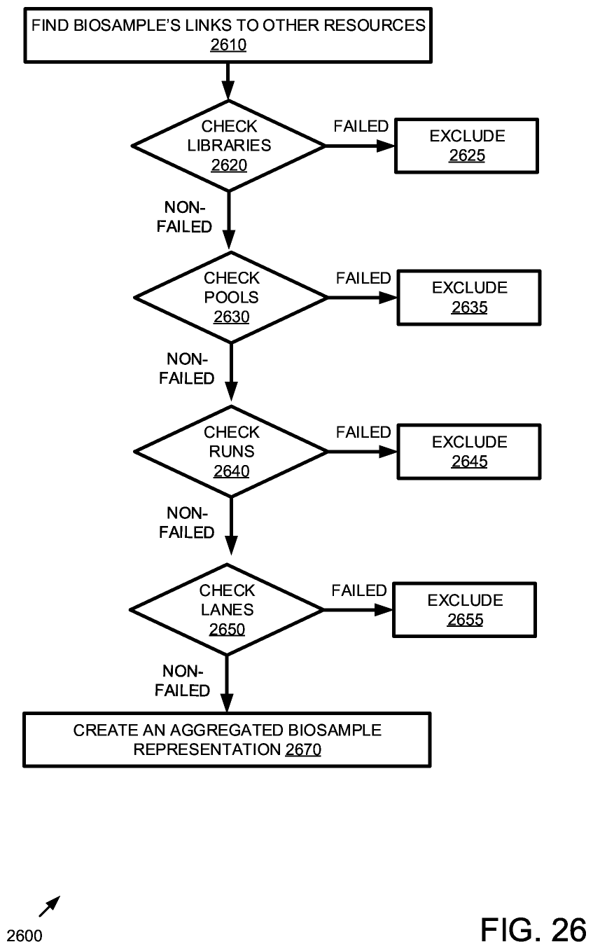

FIG. 26 is a flowchart of an example method 2600 of implementing quality-control-based selective yield aggregation across sequencing entities and can be implemented in any of the examples herein involving aggregation and quality control, including 2380 of FIG. 23. Such a method can be implemented by a quality-control-based selective aggregator for quality-control-based selective aggregation as described herein.

At 2610, a biosample is often linked to downstream entities, such as libraries, pools, runs, and flowcell lanes. Such relationships can be used to collect data when the biosample is chosen as an input.

A biosample is linked to one or more libraries, at 2620, the environment checks for any libraries set to a status of “QC Failed” and excludes them at 2625. FASTQ files coming from the library are excluded. If there are libraries that are not failed, other sequencing entities can be checked.

A biosample may be linked to one or more pools. At 2630, the environment checks for any pools that are failed and excludes them at 2635.

A biosample may be linked to one or more runs. At 2640, the environment checks for any runs that are failed and excludes them at 2645.

A biosample may be linked to one or more lanes from the same or different runs. At 2650, the environment checks for any lanes that are failed and excludes them at 2655.

The aggregator of the environment can then collect the FASTQ files coming from libraries, pools, runs, and lanes that are not set to a failure status. The files are linked to a created aggregated biosample representation in the environment.

The aggregated sample and linked FASTQ files can be used as an input to the application. The FASTQ files can be formatted for suitable consumption by the yield analysis application if desired.

### Example 55—Example Sequencing Technologies

A variety of sequencing technologies can be implemented in conjunction with the sequencing devices described herein.

Library Preparation

Libraries comprising polynucleotides may be prepared in any suitable manner to attach oligonucleotide adapters to target polynucleotides. As used herein, a “library” is a population of polynucleotides from a given source or sample. A library comprises a plurality of target polynucleotides. As used herein, a “target polynucleotide” is a polynucleotide that is desired to sequence. The target polynucleotide may be essentially any polynucleotide of known or unknown sequence. It may be, for example, a fragment of genomic DNA or cDNA. Sequencing may result in determination of the sequence of the whole, or a part of the target polynucleotides. The target polynucleotides may be derived from a primary polynucleotide sample that has been randomly fragmented. The target polynucleotides may be processed into templates suitable for amplification by the placement of universal primer sequences at the ends of each target fragment. The target polynucleotides may also be obtained from a primary RNA sample by reverse transcription into cDNA.

As used herein, the terms “polynucleotide” and “oligonucleotide” may be used interchangeably and refer to a molecule comprising two or more nucleotide monomers covalently bound to one another, typically through a phosphodiester bond. Polynucleotides typically contain more nucleotides than oligonucleotides. For purposes of illustration and not limitation, a polynucleotide may be considered to contain 15, 20, 30, 40, 50, 100, 200, 300, 400, 500, or more nucleotides, while an oligonucleotide may be considered to contain 100, 50, 20, 15 or less nucleotides.

Polynucleotides and oligonucleotides may comprise deoxyribonucleic acid (DNA) or ribonucleic acid (RNA). The terms should be understood to include, as equivalents, analogs of either DNA or RNA made from nucleotide analogs and to be applicable to single stranded (such as sense or antisense) and double stranded polynucleotides. The term as used herein also encompasses cDNA, that is complementary or copy DNA produced from an RNA template, for example by the action of reverse transcriptase.

Primary polynucleotide molecules may originate in double-stranded DNA (dsDNA) form (e.g. genomic DNA fragments, PCR and amplification products and the like) or may have originated in single-stranded form, as DNA or RNA, and been converted to dsDNA form. By way of example, mRNA molecules may be copied into double-stranded cDNAs using standard techniques well known in the art. The precise sequence of primary polynucleotides is generally not material to the disclosure presented herein, and may be known or unknown.

In some embodiments, the primary target polynucleotides are RNA molecules. In an aspect of such embodiments, RNA isolated from specific samples is first converted to double-stranded DNA using techniques known in the art. The double-stranded DNA may then be index tagged with a library specific tag. Different preparations of such double-stranded DNA comprising library specific index tags may be generated, in parallel, from RNA isolated from different sources or samples. Subsequently, different preparations of double-stranded DNA comprising different library specific index tags may be mixed, sequenced en masse, and the identity of each sequenced fragment determined with respect to the library from which it was isolated/derived by virtue of the presence of a library specific index tag sequence.

In some embodiments, the primary target polynucleotides are DNA molecules. For example, the primary polynucleotides may represent the entire genetic complement of an organism, and are genomic DNA molecules, such as human DNA molecules, which include both intron and exon sequences (coding sequence), as well as non-coding regulatory sequences such as promoter and enhancer sequences. Although it could be envisaged that particular sub-sets of polynucleotide sequences or genomic DNA could also be used, such as, for example, particular chromosomes or a portion thereof. In many embodiments, the sequence of the primary polynucleotides is not known. The DNA target polynucleotides may be treated chemically or enzymatically either prior to, or subsequent to a fragmentation processes, such as a random fragmentation process, and prior to, during, or subsequent to the ligation of the adapter oligonucleotides.

The primary target polynucleotides can be fragmented to appropriate lengths suitable for sequencing. The target polynucleotides may be fragmented in any suitable manner. The target polynucleotides can be randomly fragmented. Random fragmentation refers to the fragmentation of a polynucleotide in a non-ordered fashion by, for example, enzymatic, chemical or mechanical means. Such fragmentation methods are known in the art and utilize standard methods (Sambrook and Russell, Molecular Cloning, A Laboratory Manual, third edition). For the sake of clarity, generating smaller fragments of a larger piece of polynucleotide via specific PCR amplification of such smaller fragments is not equivalent to fragmenting the larger piece of polynucleotide because the larger piece of polynucleotide remains in intact (i.e., is not fragmented by the PCR amplification). Moreover, random fragmentation is designed to produce fragments irrespective of the sequence identity or position of nucleotides comprising and/or surrounding the break.

In some embodiments, the random fragmentation is by mechanical means such as nebulization or sonication to produce fragments of about 50 base pairs in length to about 1500 base pairs in length, such as 50-700 base pairs in length or 50-500 base pairs in length.

Fragmentation of polynucleotide molecules by mechanical means (nebulization, sonication and Hydroshear for example) may result in fragments with a heterogeneous mix of blunt and 3′- and 5′-overhanging ends. Fragment ends may be repaired using methods or kits (such as the Lucigen DNA terminator End Repair Kit) known in the art to generate ends that are optimal for insertion, for example, into blunt sites of cloning vectors. In some embodiments, the fragment ends of the population of nucleic acids are blunt ended. The fragment ends may be blunt ended and phosphorylated. The phosphate moiety may be introduced via enzymatic treatment, for example, using polynucleotide kinase.

In some embodiments, the target polynucleotide sequences are prepared with single overhanging nucleotides by, for example, activity of certain types of DNA polymerase such as Taq polymerase or Klenow exo minus polymerase which has a nontemplate-dependent terminal transferase activity that adds a single deoxynucleotide, for example, deoxyadenosine (A) to the 3′ ends of, for example, PCR products. Such enzymes may be utilized to add a single nucleotide ‘A’ to the blunt ended 3′ terminus of each strand of the target polynucleotide duplexes. Thus, an ‘A’ could be added to the 3′ terminus of each end repaired duplex strand of the target polynucleotide duplex by reaction with Taq or Klenow exo minus polymerase, while the adapter polynucleotide construct could be a T-construct with a compatible ‘T’ overhang present on the 3′ terminus of each duplex region of the adapter construct. This end modification also prevents self-ligation of the target polynucleotides such that there is a bias towards formation of the combined ligated adapter-target polynucleotides.

In some embodiments, fragmentation is accomplished through tagmentation as described in, for example, International Patent Application Publication WO 2016/130704. In such methods transposases are employed to fragment a double stranded polynucleotide and attach a universal primer sequence into one strand of the double stranded polynucleotide. The resulting molecule may be gap-filled and subject to extension, for example by PCR amplification, using primers that comprise a 3′ end having a sequence complementary to the attached universal primer sequence and a 5′ end that contains other sequences of an adapter.

The adapters may be attached to the target polynucleotide in any other suitable manner. In some embodiments, the adapters are introduced in a multi-step process, such as a two-step process, involving ligation of a portion of the adapter to the target polynucleotide having a universal primer sequence. The second step comprises extension, for example by PCR amplification, using primers that comprise a 3′ end having a sequence complementary to the attached universal primer sequence and a 5′ end that contains other sequences of an adapter. By way of example, such extension may be performed as described in U.S. Pat. No. 8,053,192. Additional extensions may be performed to provide additional sequences to the 5′ end of the resulting previously extended polynucleotide.

In some embodiments, the entire adapter is ligated to the fragmented target polynucleotide. The ligated adapter can comprise a double stranded region that is ligated to a double stranded target polynucleotide. The double-stranded region can be as short as possible without loss of function. In this context, “function” refers to the ability of the double-stranded region to form a stable duplex under standard reaction conditions. In some embodiments, standard reactions conditions refer to reaction conditions for an enzyme-catalyzed polynucleotide ligation reaction, which will be well known to the skilled reader (e.g. incubation at a temperature in the range of 4° C. to 25° C. in a ligation buffer appropriate for the enzyme), such that the two strands forming the adapter remain partially annealed during ligation of the adapter to a target molecule. Ligation methods are known in the art and may utilize standard methods (Sambrook and Russell, Molecular Cloning, A Laboratory Manual, third edition). Such methods utilize ligase enzymes such as DNA ligase to effect or catalyze joining of the ends of the two polynucleotide strands of, in this case, the adapter duplex oligonucleotide and the target polynucleotide duplexes, such that covalent linkages are formed. The adapter duplex oligonucleotide may contain a 5′-phosphate moiety in order to facilitate ligation to a target polynucleotide 3′-OH. The target polynucleotide may contain a 5′-phosphate moiety, either residual from the shearing process, or added using an enzymatic treatment step, and has been end repaired, and optionally extended by an overhanging base or bases, to give a 3′-OH suitable for ligation. In this context, attaching means covalent linkage of polynucleotide strands which were not previously covalently linked. In a particular aspect, such attaching takes place by formation of a phosphodiester linkage between the two polynucleotide strands, but other means of covalent linkage (e.g. non-phosphodiester backbone linkages) may be used. Ligation of adapters to target polynucleotides is described in more detail in, for example, U.S. Pat. No. 8,053,192.

Any suitable adapter may be attached to a target polynucleotide via any suitable process, such as those discussed above. The adapter includes a library-specific index tag sequence. The index tag sequence may be attached to the target polynucleotides from each library before the sample is immobilized for sequencing. The index tag is not itself formed by part of the target polynucleotide, but becomes part of the template for amplification. The index tag may be a synthetic sequence of nucleotides which is added to the target as part of the template preparation step. Accordingly, a library-specific index tag is a nucleic acid sequence tag which is attached to each of the target molecules of a particular library, the presence of which is indicative of or is used to identify the library from which the target molecules were isolated.

The index tag sequence can be 20 nucleotides or less in length. For example, the index tag sequence may be 1-10 nucleotides or 4-6 nucleotides in length. A four nucleotide index tag gives a possibility of multiplexing 256 samples on the same array, a six base index tag enables 4,096 samples to be processed on the same array.

The adapters may contain more than one index tag so that the multiplexing possibilities may be increased.

The adapters can comprise a double stranded region and a region comprising two non-complementary single strands. The double-stranded region of the adapter may be of any suitable number of base pairs. The double stranded region can be a short double-stranded region, typically comprising 5 or more consecutive base pairs, formed by annealing of two partially complementary polynucleotide strands. This “double-stranded region” of the adapter refers to a region in which the two strands are annealed and does not imply any particular structural conformation. In some embodiments, the double stranded region comprises 20 or less consecutive base pairs, such as 10 or less or 5 or less consecutive base pairs.

The stability of the double-stranded region may be increased, and hence its length potentially reduced, by the inclusion of non-natural nucleotides which exhibit stronger base-pairing than standard Watson-Crick base pairs. The two strands of the adapter can be 100% complementary in the double-stranded region.

When the adapter is attached to the target polynucleotide, the non-complementary single stranded region may form the 5′ and 3′ ends of the polynucleotide to be sequenced. The term “non-complementary single stranded region” refers to a region of the adapter where the sequences of the two polynucleotide strands forming the adapter exhibit a degree of non-complementarity such that the two strands are not capable of fully annealing to each other under standard annealing conditions for a PCR reaction.

The non-complementary single stranded region is provided by different portions of the same two polynucleotide strands which form the double-stranded region. The lower limit on the length of the single-stranded portion will typically be determined by function of, for example, providing a suitable sequence for binding of a primer for primer extension, PCR and/or sequencing. Theoretically there is no upper limit on the length of the unmatched region, except that in general it is advantageous to minimize the overall length of the adapter, for example, in order to facilitate separation of unbound adapters from adapter-target constructs following the attachment step or steps. Therefore, it is generally preferred that the non-complementary single-stranded region of the adapter is 50 or less consecutive nucleotides in length, such as 40 or less, 30 or less, or 25 or less consecutive nucleotides in length.

The library-specific index tag sequence may be located in a single-stranded, double-stranded region, or span the single-stranded and double-stranded regions of the adapter. The index tag sequence can be in a single-stranded region of the adapter.

The adapters may include any other suitable sequence in addition to the index tag sequence. For example, the adapters may comprise universal extension primer sequences, which are typically located at the 5′ or 3′ end of the adapter and the resulting polynucleotide for sequencing. The universal extension primer sequences may hybridize to complementary primers bound to a surface of a solid substrate. The complementary primers comprise a free 3′ end from which a polymerase or other suitable enzyme may add nucleotides to extend the sequence using the hybridized library polynucleotide as a template, resulting in a reverse strand of the library polynucleotide being coupled to the solid surface. Such extension may be part of a sequencing run or cluster amplification.

In some embodiments, the adapters comprise one or more universal sequencing primer sequences. The universal sequencing primer sequences may bind to sequencing primers to allow sequencing of an index tag sequence, a target sequence, or an index tag sequence and a target sequence.

The precise nucleotide sequence of the adapters is generally not material to the technologies and may be selected by the user such that the desired sequence elements are ultimately included in the common sequences of the library of templates derived from the adapters to, for example, provide binding sites for particular sets of universal extension primers and/or sequencing primers.

The adapter oligonucleotides may contain exonuclease resistant modifications such as phosphorothioate linkages.

The adapter can be attached to both ends of a target polypeptide to produce a polynucleotide having a first adapter-target-second adapter sequence of nucleotides. The first and second adapters may be the same or different. The first and second adapters can be the same. If the first and second adapters are different, at least one of the first and second adapters comprises a library-specific index tag sequence.

It will be understood that a “first adapter-target-second adapter sequence” or an “adapter-target-adapter” sequence refers to the orientation of the adapters relative to one another and to the target and does not necessarily mean that the sequence may not include additional sequences, such as linker sequences, for example.

Other libraries may be prepared in a similar manner, each including at least one library-specific index tag sequence or combinations of index tag sequences different than an index tag sequence or combination of index tag sequences from the other libraries.

As used herein, “attached” or “bound” are used interchangeably in the context of an adapter relative to a target sequence. As described above, any suitable process may be used to attach an adapter to a target polynucleotide. For example, the adapter may be attached to the target through ligation with a ligase; through a combination of ligation of a portion of an adapter and addition of further or remaining portions of the adapter through extension, such as PCR, with primers containing the further or remaining portions of the adapters; trough transposition to incorporate a portion of an adapter and addition of further or remaining portions of the adapter through extension, such as PCR, with primers containing the further or remaining portions of the adapters; or the like. The attached adapter oligonucleotide can be covalently bound to the target polynucleotide.

After the adapters are attached to the target polynucleotides, the resulting polynucleotides may be subjected to a clean-up process to enhance the purity to the adapter-target-adapter polynucleotides by removing at least a portion of the unincorporated adapters. Any suitable clean-up process may be used, such as electrophoresis, size exclusion chromatography, or the like. In some embodiments, solid phase reverse immobilization (SPRI) paramagnetic beads may be employed to separate the adapter-target-adapter polynucleotides from the unattached adapters. While such processes may enhance the purity of the resulting adapter-target-adapter polynucleotides, some unattached adapter oligonucleotides likely remain.

Preparation of Immobilized Samples for Sequencing

The plurality of adapter-target-adapter molecules from one or more sources are then immobilized and amplified prior to sequencing. Methods for attaching adapter-target-adapter molecules from one or more sources to a substrate are known in the art. Likewise, methods for amplifying immobilized adapter-target-adapter molecules include, but are not limited to, bridge amplification and kinetic exclusion. Methods for immobilizing and amplifying prior to sequencing are described in, for instance, Bignell et al. (U.S. Pat. No. 8,053,192), Gunderson et al. (WO2016/130704), Shen et al. (U.S. Pat. No. 8,895,249), and Pipenburg et al. (U.S. Pat. No. 9,309,502).

A sample, including pooled samples, can then be immobilized in preparation for sequencing. Sequencing can be performed as an array of single molecules, or can be amplified prior to sequencing. The amplification can be carried out using one or more immobilized primers. The immobilized primer(s) can be a lawn on a planar surface, or on a pool of beads. The pool of beads can be isolated into an emulsion with a single bead in each “compartment” of the emulsion. At a concentration of only one template per “compartment”, only a single template is amplified on each bead.

The term “solid-phase amplification” as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers is/are immobilized on the solid support. Solid phase PCR covers systems such as emulsions, wherein one primer is anchored to a bead and the other is in free solution, and colony formation in solid phase gel matrices wherein one primer is anchored to the surface, and one is in free solution.

In some embodiments, the solid support comprises a patterned surface. A “patterned surface” refers to an arrangement of different regions in or on an exposed layer of a solid support. For example, one or more of the regions can be features where one or more amplification primers are present. The features can be separated by interstitial regions where amplification primers are not present. In some embodiments, the pattern can be an x-y format of features that are in rows and columns. In some embodiments, the pattern can be a repeating arrangement of features and/or interstitial regions. In some embodiments, the pattern can be a random arrangement of features and/or interstitial regions. Exemplary patterned surfaces that can be used in the methods and compositions set forth herein are described in U.S. Pat. Nos. 8,778,848, 8,778,849 and 9,079,148, and US Pub. No. 2014/0243224, each of which is incorporated herein by reference.

In some embodiments, the solid support comprises an array of wells or depressions in a surface. This may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface can be wells in an array of wells (e.g. microwells or nanowells) on glass, silicon, plastic or other suitable solid supports with patterned, covalently-linked gel such as poly(N-(5-azidoacetamidylpentyl)acrylamide-co-acrylamide) (PAZAM, see, for example, US Pub. No. 2013/184796, WO 2016/066586, and WO 2015/002813, each of which is incorporated herein by reference in its entirety). The process creates gel pads used for sequencing that can be stable over sequencing runs with a large number of cycles. The covalent linking of the polymer to the wells is helpful for maintaining the gel in the structured features throughout the lifetime of the structured substrate during a variety of uses. However in many embodiments, the gel need not be covalently linked to the wells. For example, in some conditions silane free acrylamide (SFA, see, for example, U.S. Pat. No. 8,563,477, which is incorporated herein by reference in its entirety) which is not covalently attached to any part of the structured substrate, can be used as the gel material.

In particular embodiments, a structured substrate can be made by patterning a solid support material with wells (e.g. microwells or nanowells), coating the patterned support with a gel material (e.g. PAZAM, SFA or chemically modified variants thereof, such as the azidolyzed version of SFA (azido-SFA)) and polishing the gel coated support, for example via chemical or mechanical polishing, thereby retaining gel in the wells but removing or inactivating substantially all of the gel from the interstitial regions on the surface of the structured substrate between the wells. Primer nucleic acids can be attached to gel material. A solution of target nucleic acids (e.g. a fragmented human genome) can then be contacted with the polished substrate such that individual target nucleic acids will seed individual wells via interactions with primers attached to the gel material; however, the target nucleic acids will not occupy the interstitial regions due to absence or inactivity of the gel material. Amplification of the target nucleic acids will be confined to the wells since absence or inactivity of gel in the interstitial regions prevents outward migration of the growing nucleic acid colony. The process is conveniently manufacturable, being scalable and utilizing conventional micro- or nanofabrication methods.

Although the technologies encompass “solid-phase” amplification methods in which only one amplification primer is immobilized (the other primer usually being present in free solution), it is preferred for the solid support to be provided with both the forward and the reverse primers immobilized. In practice, there will be a ‘plurality’ of identical forward primers and/or a ‘plurality’ of identical reverse primers immobilized on the solid support, since the amplification process requires an excess of primers to sustain amplification. References herein to forward and reverse primers are to be interpreted accordingly as encompassing a ‘plurality’ of such primers unless the context indicates otherwise.

Any given amplification reaction requires at least one type of forward primer and at least one type of reverse primer specific for the template to be amplified. However, in certain embodiments the forward and reverse primers may comprise template-specific portions of identical sequence, and may have entirely identical nucleotide sequence and structure (including any non-nucleotide modifications). In other words, it is possible to carry out solid-phase amplification using only one type of primer, and such single-primer methods are encompassed within the scope of the technologies. Other embodiments may use forward and reverse primers which contain identical template-specific sequences but which differ in some other structural features. For example one type of primer may contain a non-nucleotide modification which is not present in the other.

In embodiments of the disclosure, primers for solid-phase amplification can be immobilized by single point covalent attachment to the solid support at or near the 5′ end of the primer, leaving the template-specific portion of the primer free to anneal to its cognate template and the 3′ hydroxyl group free for primer extension. Any suitable covalent attachment means known in the art may be used for this purpose. The chosen attachment chemistry will depend on the nature of the solid support, and any derivatization or functionalization applied to it. The primer itself may include a moiety, which may be a non-nucleotide chemical modification, to facilitate attachment. In a particular embodiment, the primer may include a sulphur-containing nucleophile, such as phosphorothioate or thiophosphate, at the 5′ end. In the case of solid-supported polyacrylamide hydrogels, this nucleophile will bind to a bromoacetamide group present in the hydrogel. A more particular means of attaching primers and templates to a solid support is via 5′ phosphorothioate attachment to a hydrogel comprised of polymerized acrylamide and N-(5-bromoacetamidylpentyl) acrylamide (BRAPA), as described fully in WO 05/065814.

Certain embodiments may make use of solid supports comprised of an inert substrate or matrix (e.g. glass slides, polymer beads, etc.) which has been “functionalized”, for example by application of a layer or coating of an intermediate material comprising reactive groups which permit covalent attachment to biomolecules, such as polynucleotides. Examples of such supports include, but are not limited to, polyacrylamide hydrogels supported on an inert substrate such as glass. In such embodiments, the biomolecules (e.g. polynucleotides) may be directly covalently attached to the intermediate material (e.g. the hydrogel), but the intermediate material may itself be non-covalently attached to the substrate or matrix (e.g. the glass substrate). The term “covalent attachment to a solid support” is to be interpreted accordingly as encompassing this type of arrangement.

The pooled samples may be amplified on beads wherein each bead contains a forward and reverse amplification primer. In a particular embodiment, the library of templates can be used to prepare clustered arrays of nucleic acid colonies, analogous to those described in U.S. Pub. No. 2005/0100900, U.S. Pat. No. 7,115,400, WO 00/18957 and WO 98/44151, the contents of which are incorporated herein by reference in their entirety, by solid-phase amplification and more particularly solid phase isothermal amplification. The terms ‘cluster’ and ‘colony’ are used interchangeably herein to refer to a discrete site on a solid support comprised of a plurality of identical immobilized nucleic acid strands and a plurality of identical immobilized complementary nucleic acid strands. The term “clustered array” refers to an array formed from such clusters or colonies. In this context the term “array” is not to be understood as requiring an ordered arrangement of clusters.

The term “solid phase”, or “surface”, is used to mean either a planar array wherein primers are attached to a flat surface, for example, glass, silica or plastic microscope slides or similar flow cell devices; beads, wherein either one or two primers are attached to the beads and the beads are amplified; or an array of beads on a surface after the beads have been amplified.

Clustered arrays can be prepared using either a process of thermocycling, as described in WO 98/44151, or a process whereby the temperature is maintained as a constant, and the cycles of extension and denaturing are performed using changes of reagents. Such isothermal amplification methods are described in patent application numbers WO 02/46456 and U.S. Pub. No. 2008/0009420, which are incorporated herein by reference in their entirety. Due to the lower temperatures required in the isothermal process, this is particularly preferred.

It will be appreciated that any of the amplification methodologies described herein or generally known in the art may be utilized with universal or target-specific primers to amplify immobilized DNA fragments. Suitable methods for amplification include, but are not limited to, the polymerase chain reaction (PCR), strand displacement amplification (SDA), transcription mediated amplification (TMA) and nucleic acid sequence based amplification (NASBA), as described in U.S. Pat. No. 8,003,354, which is incorporated herein by reference in its entirety. The above amplification methods may be employed to amplify one or more nucleic acids of interest. For example, PCR, including multiplex PCR, SDA, TMA, NASBA and the like may be utilized to amplify immobilized DNA fragments. In some embodiments, primers directed specifically to the polynucleotide of interest are included in the amplification reaction.

Other suitable methods for amplification of polynucleotides may include oligonucleotide extension and ligation, rolling circle amplification (RCA) (Lizardi et al., Nat. Genet. 19:225-232 (1998)) and oligonucleotide ligation assay (OLA) (See generally U.S. Pat. Nos. 7,582,420, 5,185,243, 5,679,524 and 5,573,907; EP 0 320 308 B1; EP 0 336 731 B1; EP 0 439 182 B1; WO 90/01069; WO 89/12696; and WO 89/09835) technologies. It will be appreciated that these amplification methodologies may be designed to amplify immobilized DNA fragments. For example, in some embodiments, the amplification method may include ligation probe amplification or oligonucleotide ligation assay (OLA) reactions that contain primers directed specifically to the nucleic acid of interest. In some embodiments, the amplification method may include a primer extension-ligation reaction that contains primers directed specifically to the nucleic acid of interest. As a non-limiting example of primer extension and ligation primers that may be specifically designed to amplify a nucleic acid of interest, the amplification may include primers used for the GoldenGate assay (Illumina, Inc., San Diego, Calif.) as exemplified by U.S. Pat. Nos. 7,582,420 and 7,611,869.

Exemplary isothermal amplification methods that may be used in a method of the present disclosure include, but are not limited to, Multiple Displacement Amplification (MDA) as exemplified by, for example Dean et al., Proc. Natl. Acad. Sci. USA 99:5261-66 (2002) or isothermal strand displacement nucleic acid amplification exemplified by, for example U.S. Pat. No. 6,214,587. Other non-PCR-based methods that may be used in the present disclosure include, for example, strand displacement amplification (SDA) which is described in, for example Walker et al., Molecular Methods for Virus Detection, Academic Press, Inc., 1995; U.S. Pat. Nos. 5,455,166, and 5,130,238, and Walker et al., Nucl. Acids Res. 20:1691-96 (1992) or hyper-branched strand displacement amplification which is described in, for example Lage et al., Genome Res. 13:294-307 (2003). Isothermal amplification methods may be used with the strand-displacing Phi 29 polymerase or Bst DNA polymerase large fragment, 5′->3′ exo- for random primer amplification of genomic DNA. The use of these polymerases takes advantage of their high processivity and strand displacing activity. High processivity allows the polymerases to produce fragments that are 10-20 kb in length. As set forth above, smaller fragments may be produced under isothermal conditions using polymerases having low processivity and strand-displacing activity such as Klenow polymerase. Additional description of amplification reactions, conditions and components are set forth in detail in the disclosure of U.S. Pat. No. 7,670,810, which is incorporated herein by reference in its entirety.

Another polynucleotide amplification method that is useful in the present disclosure is Tagged PCR which uses a population of two-domain primers having a constant 5′ region followed by a random 3′ region as described, for example, in Grothues et al. Nucleic Acids Res. 21(5):1321-2 (1993). The first rounds of amplification are carried out to allow a multitude of initiations on heat denatured DNA based on individual hybridization from the randomly-synthesized 3′ region. Due to the nature of the 3′ region, the sites of initiation are contemplated to be random throughout the genome. Thereafter, the unbound primers may be removed and further replication may take place using primers complementary to the constant 5′ region.

In some embodiments, isothermal amplification can be performed using kinetic exclusion amplification (KEA), also referred to as exclusion amplification (ExAmp). A nucleic acid library of the present disclosure can be made using a method that includes a step of reacting an amplification reagent to produce a plurality of amplification sites that each includes a substantially clonal population of amplicons from an individual target nucleic acid that has seeded the site. In some embodiments the amplification reaction proceeds until a sufficient number of amplicons are generated to fill the capacity of the respective amplification site. Filling an already seeded site to capacity in this way inhibits target nucleic acids from landing and amplifying at the site thereby producing a clonal population of amplicons at the site. In some embodiments, apparent clonality can be achieved even if an amplification site is not filled to capacity prior to a second target nucleic acid arriving at the site. Under some conditions, amplification of a first target nucleic acid can proceed to a point that a sufficient number of copies are made to effectively outcompete or overwhelm production of copies from a second target nucleic acid that is transported to the site. For example in an embodiment that uses a bridge amplification process on a circular feature that is smaller than 500 nm in diameter, it has been determined that after 14 cycles of exponential amplification for a first target nucleic acid, contamination from a second target nucleic acid at the same site will produce an insufficient number of contaminating amplicons to adversely impact sequencing-by-synthesis analysis on an Illumina sequencing platform.

Amplification sites in an array can be, but need not be, entirely clonal in particular embodiments. Rather, for some applications, an individual amplification site can be predominantly populated with amplicons from a first target nucleic acid and can also have a low level of contaminating amplicons from a second target nucleic acid. An array can have one or more amplification sites that have a low level of contaminating amplicons so long as the level of contamination does not have an unacceptable impact on a subsequent use of the array. For example, when the array is to be used in a detection application, an acceptable level of contamination would be a level that does not impact signal to noise or resolution of the detection technique in an unacceptable way. Accordingly, apparent clonality will generally be relevant to a particular use or application of an array made by the methods set forth herein. Exemplary levels of contamination that can be acceptable at an individual amplification site for particular applications include, but are not limited to, at most 0.1%, 0.5%, 1%, 5%, 10% or 25% contaminating amplicons. An array can include one or more amplification sites having these exemplary levels of contaminating amplicons. For example, up to 5%, 10%, 25%, 50%, 75%, or even 100% of the amplification sites in an array can have some contaminating amplicons. It will be understood that in an array or other collection of sites, at least 50%, 75%, 80%, 85%, 90%, 95% or 99% or more of the sites can be clonal or apparently clonal.

In some embodiments, kinetic exclusion can occur when a process occurs at a sufficiently rapid rate to effectively exclude another event or process from occurring. Take for example the making of a nucleic acid array where sites of the array are randomly seeded with target nucleic acids from a solution and copies of the target nucleic acid are generated in an amplification process to fill each of the seeded sites to capacity. In accordance with the kinetic exclusion methods of the present disclosure, the seeding and amplification processes can proceed simultaneously under conditions where the amplification rate exceeds the seeding rate. As such, the relatively rapid rate at which copies are made at a site that has been seeded by a first target nucleic acid will effectively exclude a second nucleic acid from seeding the site for amplification. Kinetic exclusion amplification methods can be performed as described in detail in the disclosure of US Application Pub. No. 2013/0338042, which is incorporated herein by reference in its entirety.

Kinetic exclusion can exploit a relatively slow rate for initiating amplification (e.g. a slow rate of making a first copy of a target nucleic acid) vs. a relatively rapid rate for making subsequent copies of the target nucleic acid (or of the first copy of the target nucleic acid). In the example of the previous paragraph, kinetic exclusion occurs due to the relatively slow rate of target nucleic acid seeding (e.g. relatively slow diffusion or transport) vs. the relatively rapid rate at which amplification occurs to fill the site with copies of the nucleic acid seed. In another exemplary embodiment, kinetic exclusion can occur due to a delay in the formation of a first copy of a target nucleic acid that has seeded a site (e.g. delayed or slow activation) vs. the relatively rapid rate at which subsequent copies are made to fill the site. In this example, an individual site may have been seeded with several different target nucleic acids (e.g. several target nucleic acids can be present at each site prior to amplification). However, first copy formation for any given target nucleic acid can be activated randomly such that the average rate of first copy formation is relatively slow compared to the rate at which subsequent copies are generated. In this case, although an individual site may have been seeded with several different target nucleic acids, kinetic exclusion will allow only one of those target nucleic acids to be amplified. More specifically, once a first target nucleic acid has been activated for amplification, the site will rapidly fill to capacity with its copies, thereby preventing copies of a second target nucleic acid from being made at the site.

An amplification reagent can include further components that facilitate amplicon formation and in some cases increase the rate of amplicon formation. An example is a recombinase. Recombinase can facilitate amplicon formation by allowing repeated invasion/extension. More specifically, recombinase can facilitate invasion of a target nucleic acid by the polymerase and extension of a primer by the polymerase using the target nucleic acid as a template for amplicon formation. This process can be repeated as a chain reaction where amplicons produced from each round of invasion/extension serve as templates in a subsequent round. The process can occur more rapidly than standard PCR since a denaturation cycle (e.g. via heating or chemical denaturation) is not required. As such, recombinase-facilitated amplification can be carried out isothermally. It is generally desirable to include ATP, or other nucleotides (or in some cases non-hydrolyzable analogs thereof) in a recombinase-facilitated amplification reagent to facilitate amplification. A mixture of recombinase and single stranded binding (SSB) protein is particularly useful as SSB can further facilitate amplification. Exemplary formulations for recombinase-facilitated amplification include those sold commercially as TwistAmp kits by TwistDx (Cambridge, UK). Useful components of recombinase-facilitated amplification reagent and reaction conditions are set forth in U.S. Pat. Nos. 5,223,414 and 7,399,590, each of which is incorporated herein by reference.

Another example of a component that can be included in an amplification reagent to facilitate amplicon formation and in some cases to increase the rate of amplicon formation is a helicase. Helicase can facilitate amplicon formation by allowing a chain reaction of amplicon formation. The process can occur more rapidly than standard PCR since a denaturation cycle (e.g. via heating or chemical denaturation) is not required. As such, helicase-facilitated amplification can be carried out isothermally. A mixture of helicase and single stranded binding (SSB) protein is particularly useful as SSB can further facilitate amplification. Exemplary formulations for helicase-facilitated amplification include those sold commercially as IsoAmp kits from Biohelix (Beverly, Mass.). Further, examples of useful formulations that include a helicase protein are described in U.S. Pat. Nos. 7,399,590 and 7,829,284, each of which is incorporated herein by reference.

Yet another example of a component that can be included in an amplification reagent to facilitate amplicon formation and in some cases increase the rate of amplicon formation is an origin binding protein.

Use in Sequencing

Following attachment of adaptor-target-adaptor molecules to a surface, the sequence of the immobilized and amplified adapter-target-adapter molecules is determined. Sequencing can be carried out using any suitable sequencing technique, and methods for determining the sequence of immobilized and amplified adapter-target-adapter molecules, including strand re-synthesis, are known in the art and are described in, for instance, Bignell et al. (U.S. Pat. No. 8,053,192), Gunderson et al. (WO2016/130704), Shen et al. (U.S. Pat. No. 8,895,249), and Pipenburg et al. (U.S. Pat. No. 9,309,502).

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid can be an automated process. Preferred embodiments include sequencing-by-synthesis (“SBS”) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g. A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluorophores can include fluorophores linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al. described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluorophore and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Pub. Nos. 2007/0166705, 2006/0188901, 2006/0240439, 2006/0281109, 2012/0270305, and 2013/0260372, U.S. Pat. No. 7,057,026, PCT Publication No. WO 05/065814, U.S. Patent Application Publication No. 2005/0100900, and PCT Publication Nos. WO 06/064199 and WO 07/010,251, the disclosures of which are incorporated herein by reference in their entireties.

Some embodiments can utilize detection of four different nucleotides using fewer than four different labels. For example, SBS can be performed utilizing methods and systems described in the incorporated materials of U.S. Pub. No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Pub. No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”, Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414, both of which are incorporated herein by reference, or nucleotide incorporations can be detected with zero-mode waveguides as described, for example, in U.S. Pat. No. 7,315,019, which is incorporated herein by reference, and using fluorescent nucleotide analogs and engineered polymerases as described, for example, in U.S. Pat. No. 7,405,281 and U.S. Pub. No. 2008/0108082, both of which are incorporated herein by reference. The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in U.S. Pub. Nos. 2009/0026082; 2009/0127589; 2010/0137143; and 2010/0282617, all of which are incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

The technologies described can provide for rapid and efficient detection of a plurality of target nucleic acid in parallel. Accordingly, integrated systems can be capable of preparing and detecting nucleic acids using any of a variety of techniques, including those described above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in U.S. Pub. No. 2010/0111768 and U.S. Ser. No. 13/273,666 (U.S. Pub. No. 2012/0270305), each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, Calif.) and devices described in U.S. Ser. No. 13/273,666 (U.S. Pub. No. 2012/0270305), which is incorporated herein by reference.

### Example 56—Example Computing Systems

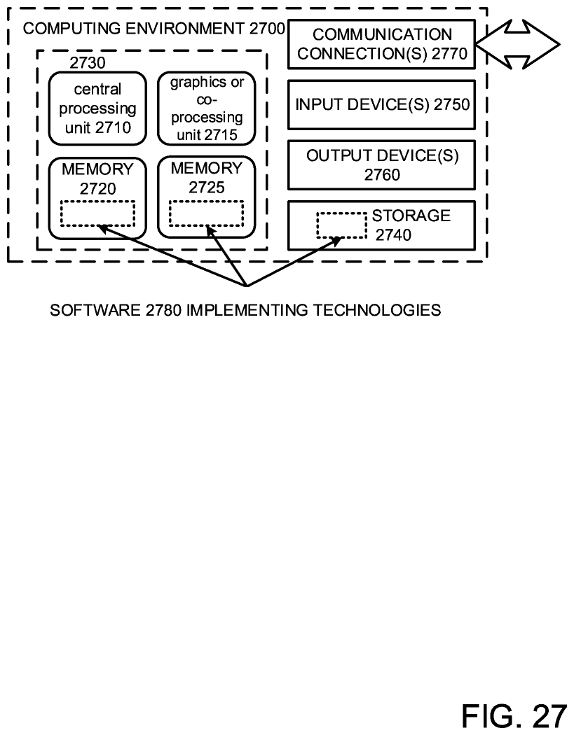

FIG. 27 illustrates a generalized example of a suitable computing system 2700 in which any of the described technologies may be implemented. The computing system 2700 is not intended to suggest any limitation as to scope of use or functionality, as the innovations may be implemented in diverse computing systems, including special-purpose computing systems. In practice, a computing system can comprise multiple networked instances of the illustrated computing system.

With reference to FIG. 27, the computing system 2700 includes one or more processing units 2710, 2715 and memory 2720, 2725. In FIG. 27, this basic configuration 2730 is included within a dashed line. The processing units 2710, 2715 execute computer-executable instructions. A processing unit can be a central processing unit (CPU), processor in an application-specific integrated circuit (ASIC), or any other type of processor. In a multi-processing system, multiple processing units execute computer-executable instructions to increase processing power. For example, FIG. 27 shows a central processing unit 2710 as well as a graphics processing unit or co-processing unit 2715. The tangible memory 2720, 2725 may be volatile memory (e.g., registers, cache, RAM), non-volatile memory (e.g., ROM, EEPROM, flash memory, etc.), or some combination of the two, accessible by the processing unit(s). The memory 2720, 2725 stores software 2780 implementing one or more innovations described herein, in the form of computer-executable instructions suitable for execution by the processing unit(s).

A computing system may have additional features. For example, the computing system 2700 includes storage 2740, one or more input devices 2750, one or more output devices 2760, and one or more communication connections 2770. An interconnection mechanism (not shown) such as a bus, controller, or network interconnects the components of the computing system 2700. Typically, operating system software (not shown) provides an operating environment for other software executing in the computing system 2700, and coordinates activities of the components of the computing system 2700.

The tangible storage 2740 may be removable or non-removable, and includes magnetic disks, magnetic tapes or cassettes, CD-ROMs, DVDs, or any other medium which can be used to store information in a non-transitory way and which can be accessed within the computing system 2700. The storage 2740 stores instructions for the software 2780 implementing one or more innovations described herein.

The input device(s) 2750 may be a touch input device such as a keyboard, mouse, pen, or trackball, a voice input device, a scanning device, or another device that provides input to the computing system 2700. For video encoding, the input device(s) 2750 may be a camera, video card, TV tuner card, or similar device that accepts video input in analog or digital form, or a CD-ROM or CD-RW that reads video samples into the computing system 2700. The output device(s) 2760 may be a display, printer, speaker, CD-writer, or another device that provides output from the computing system 2700.

The communication connection(s) 2770 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the general context of computer-executable instructions, such as those included in program modules, being executed in a computing system on a target real or virtual processor. Generally, program modules include routines, programs, libraries, objects, classes, components, data structures, etc. that perform particular tasks or implement particular abstract data types. The functionality of the program modules may be combined or split between program modules as desired in various embodiments. Computer-executable instructions for program modules may be executed within a local or distributed computing system.

For the sake of presentation, the detailed description uses terms like “determine” and “use” to describe computer operations in a computing system. These terms are high-level abstractions for operations performed by a computer, and should not be confused with acts performed by a human being. The actual computer operations corresponding to these terms vary depending on implementation.

### Example 57—Computer-Readable Media

Any of the computer-readable media herein can be non-transitory (e.g., volatile memory such as DRAM or SRAM, nonvolatile memory such as magnetic storage, optical storage, or the like) and/or tangible. Any of the storing actions described herein can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

### Example 58—Computer-Executable Implementations

Although some method acts shown relate to laboratory activities and are performed by human activity (e.g., “Prepare libraries from biosamples,”), the other acts of any the methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

Such acts of the methods described herein can be implemented by computer-executable instructions in (e.g., stored on, encoded on, or the like) one or more computer-readable media (e.g., computer-readable storage media or other tangible media) or one or more computer-readable storage devices (e.g., memory, magnetic storage, optical storage, or the like). Such instructions can cause a computing device to perform the method. The technologies described herein can be implemented in a variety of programming languages.

In any of the technologies described herein, the illustrated actions can be described from alternative perspectives while still implementing the technologies. For example, “receiving” can also be described as “sending” for a different perspective.

Further Description

Any of the following embodiments can be implemented.

Clause 1. A sequencing device system comprising:

a plurality of sequencing devices that output multiplexed raw biosample sequencing data for a plurality of input biosamples comprising a particular biosample, wherein a target number of base pairs of sequence yield is specified as sufficient for launching an application for further analysis of the particular biosample;

one or more processors; and

memory coupled to the one or more processors, wherein the memory comprises computer-executable instructions causing the one or more processors to perform a process comprising:

receiving, from the plurality of sequencing devices, the multiplexed raw biosample sequencing data for the plurality of input biosamples;

demultiplexing and converting the multiplexed raw biosample sequencing data into a plurality of candidate biosample sequencing yield data sets;

identifying which of the candidate biosample sequencing yield data sets originates from the particular biosample;

aggregating the candidate biosample sequencing yield data sets originating from the particular biosample into aggregated sequencing data yield for the particular biosample;

determining whether the aggregated sequencing data yield for the particular biosample is sufficient, wherein determining whether the aggregating sequencing data yield is sufficient comprises comparing a number of base pairs in the aggregated sequencing data yield for the particular biosample to the target number of base pairs; and

responsive to determining that the aggregated sequencing data yield for the particular biosample is sufficient, launching an application performing the further analysis of the particular biosample with the aggregated sequencing data yield for the particular biosample.

Clause 2. The sequencing device system of Clause 1 wherein the process further comprises:

identifying a portion of the candidate biosample sequencing yield data sets as failing a quality control metric; and

responsive to determining that the portion of data sets failed the quality control metric, excluding the portion of data sets from aggregation.

Clause 3. The sequencing device system of Clause 1 wherein the process further comprises:

identifying a portion of the candidate biosample sequencing yield data sets as failing a quality control metric; and

responsive to determining that the portion of the data sets failed the quality control metric, indicating the portion of data sets as failed;

via user input, receiving an override of determination that the portion of data sets failed the quality control metric; and

responsive to receiving the override, including the portion of data sets in aggregation.

Clause 4. The sequencing device system of any of Clauses 2 or 3 wherein:

identifying a portion of the candidate biosample sequencing yield data sets as failing a quality control metric comprises comparing an observed quality control metric value for a particular data set of the candidate data sets to a stored threshold value for the quality control metric.

Clause 5. The sequencing device system of Clause 4 wherein:

identifying the portion as failing the quality control metric comprises, for a particular sequencing run performed by a particular sequencing device out of the sequencing devices, identifying a sequencing lane of the sequencing device as failing the quality control metric; and

excluding the portion from aggregation comprises excluding any biosample sequencing data for the sequencing lane from aggregation.

Clause 6. The sequencing device system of Clause 5 wherein:

the excluding excludes candidate biosample sequencing yield data sets for the sequencing lane from aggregation for a plurality of biosamples.

Clause 7. The sequencing device system of any of Clauses 2-6 wherein the process further comprises:

further responsive to determining that the portion of data failed the quality control metric, updating a yield status for the particular biosample to indicate that the excluded portion failed.

Clause 8. The sequencing device system of any of Clauses 2-7 wherein the process further comprises:

responsive to determining that there is insufficient yield according to the target number of base pairs, raising a missing yield alert.

Clause 9. The sequencing device system of Clause 8 wherein:

the missing yield alert comprises a user interface element for requesting a requeue of sequence processing for the particular biosample.

Clause 10. The sequencing device system of Clause 8 or 9 wherein:

determining that there is insufficient yield comprises including yield-in-progress for the particular biosample.

Clause 11. The sequencing device system of any of Clauses 2-10 wherein the process further comprises:

after excluding the portion of biosample sequencing data from aggregation, receiving an indication to requeue a request for yield for the particular biosample;

requeuing the request for yield; and

updating a yield status for the particular biosample to reflect the requeued request for yield for the particular biosample.

Clause 12. The sequencing device system of Clause 11 wherein the process further comprises:

responsive to a request for yield status for the particular biosample, indicating an acquired yield for the particular biosample, and a yield-in-progress for the particular biosample.

Clause 13. The sequencing device system of any of Clauses 11-12 wherein the process further comprises:

including yield expected from the requeued request for yield in calculations for determining whether enough yield has been requested for the particular biosample.

Clause 14. The sequencing device system of any of Clauses 11-13 wherein the process further comprises:

including yield expected from in-progress demultiplexing or format conversion in calculations for determining whether enough yield has been requested for the particular biosample.

Clause 15. The sequencing device system of any of Clauses 11-14 wherein the process further comprises:

setting a timeout for the requeued request for yield; and

after the timeout expires, updating the yield status to indicate that the requeued request for yield has expired.

Clause 16. The sequencing device system of Clause 15 wherein:

the timeout is set for a particular sequencing run responsive to determining that yield from any lane associated with the particular sequencing run has been received.

Clause 17. The sequencing device system of any of Clauses 11-16 wherein the process further comprises:

integrating the requeued request for yield into a laboratory information management system;

receiving an indication from the laboratory information management system that the requeued request for yield has completed; and

responsive to receiving the indication that the requeued request for yield has completed, marking the requeued request as acknowledged.

Clause 18. The sequencing device system of any of Clauses 11-17 wherein the process further comprises:

tracking the requeued request for yield, wherein tracking comprises matching the requeued request for yield to an active sequencing run; and

including predicted yield from the active sequencing run in yield-in-progress for the particular biosample.

Clause 19. The sequencing device system of Clause 18 wherein:

matching the requeued request to an active run prioritizes requeues over initial requests.

Clause 20. The sequencing device system of any of Clauses 1-19 wherein:

identifying which of the candidate biosample sequencing data sets originates from the particular biosample comprises:

matching an index identifier associated with the particular biosample with respective index identifiers indicated in the candidate biosample sequencing data sets.

Clause 21. The sequencing device system of Clause 20 wherein:

the index identifier associated with the particular biosample indicates an index sequence attached to the particular biosample and read by one of the sequencing devices.

Clause 22. The sequencing device system of any of Clauses 20-21 wherein:

the index identifier is associated with the particular biosample in a sample sheet provided as part of a sequencing run for the particular biosample; and

the sample sheet indicates a biosample identifier of the particular biosample.

Clause 23. The sequencing device system of any of Clauses 20-22 wherein:

the index identifier is associated with the particular biosample in a sample sheet generated based on information provided by a laboratory information system for a sequencing run for the particular biosample; and

the sample sheet indicates a biosample identifier of the particular biosample.

Clause 24. A computer-implemented method comprising:

receiving, from a plurality of sequencing devices, multiplexed raw biosample sequencing data for a plurality of biosamples;

demultiplexing and converting the multiplexed raw biosample sequencing data into a plurality of candidate biosample sequencing yield data sets;

identifying which of the candidate biosample sequencing yield data sets originates from a particular biosample;

aggregating the candidate biosample sequencing yield data sets into aggregated sequencing data yield for the particular biosample;

determining whether the aggregated sequencing data yield for the particular biosample is sufficient, wherein determining whether the aggregating sequencing data yield is sufficient comprises comparing a number of base pairs in the aggregated sequencing data yield for the particular biosample to a target number of base pairs for the particular biosample; and

responsive to determining that the aggregated sequencing data yield for the particular biosample is sufficient, launching an application performing further analysis of the particular biosample with the aggregated sequencing data yield for the particular biosample.

Clause 25. One or more computer-readable media having encoded thereon computer-executable instructions that when executed cause a computing system to perform the method of Clause 24.

Clause 26. The method of Clause 24 further comprising:

identifying a portion of the candidate biosample sequencing yield data sets as failing a quality control metric; and

responsive to determining that the portion of data sets failed the quality control metric, excluding the portion of data sets from aggregation.

Clause 27. The method of Clause 24 or 26 further comprising:

identifying a portion of the candidate biosample sequencing yield data sets as failing a quality control metric; and

responsive to determining that the portion of the data sets failed the quality control metric, indicating the portion of data sets as failed;

via user input, receiving an override of determination that the portion of data sets failed the quality control metric; and

responsive to receiving the override, including the portion of data sets in aggregation.

Clause 28. The method of Clause 26 wherein:

identifying a portion of the candidate biosample sequencing yield data sets as failing a quality control metric comprises comparing an observed quality control metric value for a particular data set of the candidate data sets to a stored threshold value for the quality control metric.

Clause 29. The method of Clause 28 wherein:

identifying the portion as failing the quality control metric comprises, for a particular sequencing run performed by a particular sequencing device out of the sequencing devices, identifying a sequencing lane of the sequencing device as failing the quality control metric; and

excluding the portion from aggregation comprises excluding any biosample sequencing data for the sequencing lane from aggregation.

Clause 30. The method of Clause 24 or any of Clauses 26-29 further wherein:

identifying which of the candidate biosample sequencing data sets originates from the particular biosample comprises:

matching an index identifier associated with the particular biosample with respective index identifiers indicated in the candidate biosample sequencing data sets.

Clause 31. A computer-implemented method comprising:

in a computer-readable medium, storing a relationship between an index identifier of an index sequence and a biosample identifier of a particular biosample;

receiving, from a plurality of sequencing devices, multiplexed raw biosample sequencing data for a plurality of biosamples;

demultiplexing and converting the multiplexed raw biosample sequencing data into a plurality of candidate biosample sequencing yield data sets;

identifying which of the candidate biosample sequencing yield data sets originates from the particular biosample, wherein the identifying comprises matching an index identifier of an index sequence indicated in a particular candidate biosample sequencing yield data set with the index identifier stored in the relationship; and

aggregating the candidate biosample sequencing yield data sets identified as originating from the particular biosample into aggregated sequencing data yield for the particular biosample.

Clause 32. The method of Clause 31 wherein the identifying further comprises:

in the computer-readable medium, storing a relationship between a run identifier and the biosample identifier;

wherein the identifying comprises matching a run identifier of a particular candidate biosample sequencing yield data set with the run identifier stored in the relationship.

Clause 33. The method of Clause 32 wherein the identifying further comprises:

in the computer-readable medium, storing a relationship between a lane identifier and the biosample identifier;

wherein the identifying comprises matching a lane identifier of a particular candidate biosample sequencing yield data set with the lane identifier stored in the relationship.

Clause 34. A sequencing device system comprising:

a plurality of sequencing devices that output multiplexed raw biosample sequencing data for a plurality of input biosamples comprising a particular biosample;

in one or more computer-readable media, internal representations of sequencing runs, lanes, libraries, and biosamples stored as run identifiers, lane identifiers, library identifiers, and biosample identifiers; and

a yield aggregator configured to receive a demultiplexed candidate biosample sequencing yield data set originating from the multiplexed raw biosample sequencing data, determine, from the internal representations, that the data set originates from the particular biosample, aggregate the data set with other data sets originating from a same particular biosample, and provide an indication of total amount of yield acquired for the particular biosample.

Clause 100. In a sequencing environment comprising a plurality of sequencing instruments, performing the method (or process) of any of the preceding Clauses.

Clause 101. A computing system comprising:

one or more processors;

memory comprising computer-executable instructions causing the one or more processors to perform the method (or process) of any of the preceding Clauses.

Clause 102. One or more computer-readable media comprising computer-executable instructions causing a computing system to perform the method (or process) of any of the preceding Clauses.

Further Implementations

In a sequencing environment comprising a plurality of sequencing instruments, any of the methods or processes described herein can be performed.

A computing system comprising:

one or more processors;

memory comprising computer-executable instructions causing the one or more processors to perform any of the methods or processes described herein.

One or more computer-readable media comprising computer-executable instructions causing a computing system to perform any of the methods or processes described herein.

Alternatives

The technologies from any example can be combined with the technologies described in any one or more of the other examples. In view of the many possible embodiments to which the principles of the disclosed technology may be applied, it should be recognized that the illustrated embodiments are examples of the disclosed technology and should not be taken as a limitation on the scope of the disclosed technology. Rather, the scope of the disclosed technology includes what is covered by the following claims. All that comes within the scope and spirit of the claims is therefore claimed.

1. A sequencing device system comprising:
a plurality of sequencing devices that output multiplexed raw biosample sequencing data for a plurality of input biosamples comprising a particular biosample, wherein a target number of base pairs of sequence yield is specified as sufficient for launching an application for further analysis of the particular biosample;
one or more processors; and
memory coupled to the one or more processors, wherein the memory comprises computer-executable instructions causing the one or more processors to perform a process comprising:
receiving, from the plurality of sequencing devices, the multiplexed raw biosample sequencing data for the plurality of input biosamples;
demultiplexing and converting the multiplexed raw biosample sequencing data into a plurality of candidate bio sample sequencing yield data sets;
identifying which of the candidate biosample sequencing yield data sets originates from the particular biosample;
aggregating the candidate biosample sequencing yield data sets originating from the particular biosample into aggregated sequencing data yield for the particular biosample;
determining whether the aggregated sequencing data yield for the particular biosample is sufficient, wherein determining whether the aggregating sequencing data yield is sufficient comprises comparing a number of base pairs in the aggregated sequencing data yield for the particular biosample to the target number of base pairs; and
responsive to determining that the aggregated sequencing data yield for the particular biosample is sufficient, launching an application performing the further analysis of the particular biosample with the aggregated sequencing data yield for the particular biosample.

2. The sequencing device system of claim 1 wherein the process further comprises:
identifying a portion of the candidate biosample sequencing yield data sets as failing a quality control metric; and
responsive to determining that the portion of data sets failed the quality control metric, excluding the portion of data sets from aggregation.

3. The sequencing device system of claim 1 wherein the process further comprises:
identifying a portion of the candidate biosample sequencing yield data sets as failing a quality control metric; and
responsive to determining that the portion of the data sets failed the quality control metric, indicating the portion of data sets as failed;
via user input, receiving an override of determination that the portion of data sets failed the quality control metric; and
responsive to receiving the override, including the portion of data sets in aggregation.

4. The sequencing device system of claim 2 wherein:
identifying a portion of the candidate biosample sequencing yield data sets as failing a quality control metric comprises comparing an observed quality control metric value for a particular data set of the candidate data sets to a stored threshold value for the quality control metric.

5. The sequencing device system of claim 4 wherein:
identifying the portion as failing the quality control metric comprises, for a particular sequencing run performed by a particular sequencing device out of the sequencing devices, identifying a sequencing lane of the sequencing device as failing the quality control metric; and
excluding the portion from aggregation comprises excluding any biosample sequencing data for the sequencing lane from aggregation.

6. The sequencing device system of claim 5 wherein:
the excluding excludes candidate biosample sequencing yield data sets for the sequencing lane from aggregation for a plurality of biosamples.

7. The sequencing device system of claim 2 wherein the process further comprises:
further responsive to determining that the portion of data failed the quality control metric, updating a yield status for the particular biosample to indicate that the excluded portion failed.

8. The sequencing device system of claim 2 wherein the process further comprises:
responsive to determining that there is insufficient yield according to the target number of base pairs, raising a missing yield alert.

9. The sequencing device system of claim 8 wherein:
the missing yield alert comprises a user interface element for requesting a requeue of sequence processing for the particular biosample.

10. The sequencing device system of claim 8 wherein:
determining that there is insufficient yield comprises including yield-in-progress for the particular biosample.

11. The sequencing device system of claim 2 wherein the process further comprises:
after excluding the portion of biosample sequencing data from aggregation, receiving an indication to requeue a request for yield for the particular biosample;
requeuing the request for yield; and
updating a yield status for the particular biosample to reflect the requeued request for yield for the particular biosample.

12. The sequencing device system of claim 11 wherein the process further comprises:
responsive to a request for yield status for the particular biosample, indicating an acquired yield for the particular biosample, and a yield-in-progress for the particular biosample.

13. The sequencing device system of claim 11 wherein the process further comprises:
including yield expected from the requeued request for yield in calculations for determining whether enough yield has been requested for the particular biosample.

14. The sequencing device system of claim 11 wherein the process further comprises:
including yield expected from in-progress demultiplexing or format conversion in calculations for determining whether enough yield has been requested for the particular biosample.

15. The sequencing device system of claim 11 wherein the process further comprises:
setting a timeout for the requeued request for yield; and
after the timeout expires, updating the yield status to indicate that the requeued request for yield has expired.

16. (canceled)

17. The sequencing device system of claim 11 wherein the process further comprises:
integrating the requeued request for yield into a laboratory information management system;
receiving an indication from the laboratory information management system that the requeued request for yield has completed; and
responsive to receiving the indication that the requeued request for yield has completed, marking the requeued request as acknowledged.

18. (canceled)

19. (canceled)

20. The sequencing device system of claim 1 wherein:
identifying which of the candidate biosample sequencing data sets originates from the particular biosample comprises:
matching an index identifier associated with the particular biosample with respective index identifiers indicated in the candidate biosample sequencing data sets.

21. The sequencing device system of claim 20 wherein:
the index identifier associated with the particular biosample indicates an index sequence attached to the particular biosample and read by one of the sequencing devices.

22. (canceled)

23. (canceled)

24. A computer-implemented method comprising:
receiving, from a plurality of sequencing devices, multiplexed raw biosample sequencing data for a plurality of biosamples;
demultiplexing and converting the multiplexed raw biosample sequencing data into a plurality of candidate bio sample sequencing yield data sets;
identifying which of the candidate biosample sequencing yield data sets originates from a particular biosample;
aggregating the candidate biosample sequencing yield data sets into aggregated sequencing data yield for the particular biosample;
determining whether the aggregated sequencing data yield for the particular biosample is sufficient, wherein determining whether the aggregating sequencing data yield is sufficient comprises comparing a number of base pairs in the aggregated sequencing data yield for the particular biosample to a target number of base pairs for the particular biosample; and
responsive to determining that the aggregated sequencing data yield for the particular biosample is sufficient, launching an application performing further analysis of the particular biosample with the aggregated sequencing data yield for the particular biosample.

25. (canceled)

26. (canceled)

27. (canceled)

28. (canceled)

29. (canceled)

30. (canceled)

31. (canceled)

32. (canceled)

33. (canceled)

34. A sequencing device system comprising:
a plurality of sequencing devices that output multiplexed raw biosample sequencing data for a plurality of input biosamples comprising a particular biosample;
in one or more computer-readable media, internal representations of sequencing runs, lanes, libraries, and biosamples stored as run identifiers, lane identifiers, library identifiers, and biosample identifiers; and
a yield aggregator configured to receive a demultiplexed candidate biosample sequencing yield data set originating from the multiplexed raw biosample sequencing data, determine, from the internal representations, that the data set originates from the particular biosample, aggregate the data set with other data sets originating from a same particular biosample, and provide an indication of total amount of yield acquired for the particular biosample.
